(19) United States
(12) Patent Application Publication
(10) Pub. No.: US 2024/0141330 A1
(43) Pub. Date: May 2, 2024

(54) METHODS AND COMPOSITIONS FOR MUTAGENESIS SCREENING IN MAMMALIAN CELLS

(71) Applicant: THE BOARD OF REGENTS OF THE UNIVERSITY OF TEXAS SYSTEM, Austin, TX (US)

(72) Inventors: Ralf KITTLER, Dallas, TX (US); John D. MINNA, Dallas, TX (US); Paul M. YENERALL, Dallas, TX (US)

(73) Assignee: THE BOARD OF REGENTS OF THE UNIVERSITY OF TEXAS SYSTEM, Austin, TX (US)

(21) Appl. No.: 18/409,612

(22) Filed: Jan. 10, 2024

Related U.S. Application Data
(63) Continuation-in-part of application No. 18/280,803, filed on Sep. 7, 2023.
(60) Provisional application No. 63/158,723, filed on Mar. 9, 2021.

(51) Int. Cl. C12N 15/10; G01N 33/50

(52) U.S. Cl. CPC C12N 15/1082; G01N 33/5011

(57) ABSTRACT

Disclosures herein are directed to methods and compositions for the detection of and screening for mutations that convey phenotypic properties in a protein such as, for example, drug-resistance. Embodiments of the present disclosure include lentiviral-based compositions for enhanced mutagenesis and screening of genes encoding proteins of interest that confer drug resistance according to methods disclosed herein.

### CROSS REFERENCE TO RELATED APPLICATIONS

This application is a Continuation-in-Part of U.S. Non-Provisional application Ser. No. 18/280,803, entitled “METHODS AND COMPOSITIONS FOR MUTAGENESIS SCREENING IN MAMMALIAN CELLS” and filed on Sep. 7, 2023, which is a national stage application filed under 35 U.S.C. § 371 of International Patent Application No. PCT/US2022/019260 entitled “METHODS AND COMPOSITIONS FOR MUTAGENESIS SCREENING IN MAMMALIAN CELLS” and filed on Mar. 8, 2022, which claims the benefit of U.S. Provisional Application No. 63/158,723 entitled “METHODS AND COMPOSITIONS FOR MUTAGENESIS SCREENING IN MAMMALIAN CELLS” and filed on Mar. 9, 2021, the disclosures of which are hereby incorporated by reference in their entireties.

### ACKNOWLEDGEMENT OF GOVERNMENT SUPPORT

This invention was made with Government support under Grant No. CA070907 awarded by the National Institutes of Health (NIH). The government has certain rights in this invention.

### INCORPORATION BY REFERENCE OF SEQUENCE LISTING PROVIDED ELECTRONICALLY

An electronic version of the Sequence Listing is filed herewith, the contents of which are incorporated by reference in their entirety. The electronic file is 22400 bytes in size, and titled 106546-771266.xml.

### BACKGROUND

### 1. Field

The present inventive concept is directed to methods and compositions for the detection of and screening for mutations that convey any novel phenotypic property to a protein such as, for example, drug-resistance.

### 2. Discussion of Related Art

Drug resistance is an almost inevitable consequence of clinical treatments, such as cancer therapies. Although a drug may kill some cancer cells, almost invariably a subset of cancer cells will become resistant and survive the treatment. Drug-treatment resistance in cancer and other diseases ultimately proves fatal for the majority of patients affected. Studies of drug resistance has focused on identifying genetic mechanisms, such as mutations that alter protein binding of a drug for example. In trying to measure changes in drug efficiency, much of this research has focused on known mutations and not on identifying novel mutations preemptively. As such, there is a need in the field for screening platforms that can spontaneously generate mutations which introduce a drug-resistant phenotype in a target protein.

### SUMMARY OF THE INVENTION

In an aspect, the current disclosure encompasses a method of screening for one or more mutations in a nucleic acid sequence encoding a protein in a target cell; wherein the mutation conveys a phenotypic property to the protein, comprising: (a) transducing the target cell with at least one lentiviral particle comprising a pol gene nucleic acid sequence encoding a reverse transcriptase sequence comprising a M230I and a Y501W mutation with reference to SEQ ID NO: 1; (b) contacting the target cell with a biomolecule after step (a), to select for the target cell with the phenotypic property in a target cell population; (c) obtaining the nucleic acid sequence from the target cell with the phenotypic property; and (d) screening for one or more mutations that convey the phenotypic property, wherein screening comprises comparing the nucleic acid sequence encoding the protein from the target cell with the phenotypic property, to a control nucleic acid sequence, wherein the control nucleic acid encodes the protein without the one or more mutations. In an aspect, the reverse transcriptase comprises an amino acid sequence as set forth in SEQ ID NO: 14, or a sequence at least about 80% identical thereto. The nucleic acid sequence encoding the protein may be a genomic nucleic acid sequence or a heterologous nucleic acid sequence. In an aspect, the lentiviral particle further comprises the heterologous nucleic acid sequence. In an aspect, the lentiviral particle further comprises an inducible promoter operatively linked to the heterologous nucleic acid sequence. In an aspect, the inducible promoter is a tetracycline-regulated promoter.

In an aspect of a method of the current disclosure, the nucleic acid sequence encoding for the protein may encode for 2B4, 4-1BB, 4-1BBL, A33, adenosine A2a receptor, Akt, ALK, Androgen receptor, Ang-1, Ang-2, Annexin A3, Aurora A, Aurora B, B7-H3, B7-H4, Bcl-2, Bcr-Abl, BRAF, BTK, BTLA, BTN2A1, CA-125, CAIX, CCR4, CD105/endoglin, CD109, CD123, CD155, CD16, CD160, CD19, CD20, CD200, CD200R, CD22, CD24, CD25, CD27, CD28, CD30, CD33, CD36, CD37, CD38, CD40, CD40L, CD47, CD48, CD52, CD70, CD79b, CD80, CD86, CD96, CDK4, CDK6, CDK9, CEA, CEACAM1, ChK1, ChK2, c-KIT, c-Met/HGFR, COX2, CSF-1R, CSF2, CTLA-4, CXCR2, CXCR4, DDR2, DLL3, DLL4, DNAM-1, DR5, EGFR, EpCAM, EPHA3, EphB4, ERK1, ERK2/p38 MAPK, FAK, FAP, FGF-2, FGFR1, FGFR2, FGFR3, FGFR4, Flt-3, Gal-9, GITR, GITRL, Glypican-3, HDAC1, HDAC2, HDAC3, HDAC4, HDAC5, HDAC7, HDAC8, HDAC9, HER2, HER3, HER4/ERBB4, HGF, HHLA2, HIF-1α, HSP27, HSP90, HVEM, ICOS, ICOS Ligand, IDO, IGF1R, IL-13, IL-6, JAK1, JAK2, JAK3, KRAS, LAG-3, LIGHT, MDM2, MEK1, MEK2, MMP-1, MMP-10, MMP-11, MMP-13, MMP-2, MMP-7, MMP-9, mTOR, Mucin 1, Myc, NF-κB, NKG2A, NRAS, NTRK1, NTRK2, NTRK3, OX40, OX40L, p53, PAF, PARP1, PARP2, PD1, PDGFR-α, PDGFR-β, PD-L1, PD-L2, PI3Kα, PI3Kβ, PI3Kγ, PI3Kδ, PIM1, PIM3, PSMA, PTEN, RAF-1, RANKL, RET, S100A4, SIRPα, SLAMF7, SMO, Src, STAT3, STEAP-1, Syk, TDO, TGFβ, Tie-2, TIGIT, TIM-3, TLR8, TMIGD2, TNF-α, Toll-like receptor 3, TRAIL, TRAILR1, TROP-2, VEGF, VEGF-C, VEGFR-1, VEGFR-2, VEGFR-3, VISTA, γ-secretase, or any combination thereof.

In some aspects, the mutation may comprise a substitution of a base, an insertion of a base, an insertion of one or more bases, a deletion of one or more bases, or any combination thereof. The substitution of a base may comprise substituting a C for a G, an A for a C, a T for a G, a G for a C, an A for a T, a T for an A, a G for a T, a T for a C, an A for a G, a C for an A, a C for a T, a G for an A, or any combination thereof. The mutation may comprise an indel.

In an aspect of the method, the phenotypic property may be target cell viability in the presence of the biomolecule. In an aspect, the method of screening may include contacting the target cell with a biomolecule after step (a). Examples of a biomolecule include a peptide, a protein, an enzyme, an antibody, an aptamer, DNA, RNA, siRNA, an oligonucleotide, a small molecule, or any combination thereof.

In an aspect, the method of screening may include sequencing of the nucleic acid. Sequence can be obtained by any method known in the art including Sanger sequencing, pyrosequencing, reversible terminator chemistry, sequencing by ligation, H+ Ion sensitive transistor, nanopore sequencing, next generation sequencing, or any combination thereof.

In an aspect, the current disclosure also encompasses a composition for screening of one or more mutations conveying a phenotypic property in a protein comprising, at least a lentiviral particle comprising: (a) a pol gene nucleic acid sequence encoding a reverse transcriptase sequence comprising a M230I and a Y501W mutation with reference to SEQ ID NO: 1; and (b) a heterologous nucleic acid sequence encoding the protein. The reverse transcriptase may comprise an amino acid sequence as set forth in SEQ ID NO: 14, or a sequence at least about 80% identical thereto.

In an aspect the current disclosure also encompasses a method of screening for one or more genomic mutations in a target cell, wherein the one or more genomic mutations confers drug-resistance to a drug, to the target cell, the method comprising: (a) transducing the target cell with at least one lentiviral particle comprising a pol gene nucleic acid sequence encoding a reverse transcriptase sequence comprising a M230I and a Y501W mutation with reference to SEQ ID NO: 1; (b) contacting the target cell with the drug after step (a), to select for the target cell with the drug resistance, in a target cell population; (c) obtaining the genomic sequence from the target cell with drug resistance; (d) screening for one or more mutations that confer the drug resistance, wherein screening comprises comparing the genomic sequence of the target cell with the drug resistance, to a control genomic sequence obtained from a cell without drug resistance. The reverse transcriptase may comprise an amino acid sequence as set forth in SEQ ID NO: 14, or a sequence at least about 80% identical thereto. In an aspect, the drug may comprise a drug for the treatment of a cancer, including breast cancer, prostate cancer, lymphoma, skin cancer, pancreatic cancer, colon cancer, melanoma, malignant melanoma, ovarian cancer, brain cancer, primary brain carcinoma, head-neck cancer, glioma, glioblastoma, liver cancer, bladder cancer, non-small cell lung cancer, head or neck carcinoma, breast carcinoma, ovarian carcinoma, lung carcinoma, small-cell lung carcinoma, Wilms' tumor, cervical carcinoma, testicular carcinoma, bladder carcinoma, pancreatic carcinoma, stomach carcinoma, colon carcinoma, prostatic carcinoma, genitourinary carcinoma, thyroid carcinoma, esophageal carcinoma, myeloma, multiple myeloma, adrenal carcinoma, renal cell carcinoma, endometrial carcinoma, adrenal cortex carcinoma, malignant pancreatic insulinoma, malignant carcinoid carcinoma, choriocarcinoma, mycosis fungoides, malignant hypercalcemia, cervical hyperplasia, leukemia, acute lymphocytic leukemia, chronic lymphocytic leukemia, acute myelogenous leukemia, chronic myelogenous leukemia, chronic granulocytic leukemia, acute granulocytic leukemia, hairy cell leukemia, neuroblastoma, rhabdomyosarcoma, Kaposi's sarcoma, polycythemia vera, essential thrombocytosis, Hodgkin's disease, non-Hodgkin's lymphoma, soft-tissue sarcoma, mesothelioma, osteogenic sarcoma, primary macroglobulinemia, retinoblastoma, or any combination thereof.

In an aspect, the one or more mutation to be screened using the method disclosed herein may be in a gene encoding a drug target protein, for example, 2B4, 4-1BB, 4-1 BBL, A33, adenosine A2a receptor, Akt, ALK, Androgen receptor, Ang-1, Ang-2, Annexin A3, Aurora A, Aurora B, B7-H3, B7-H4, Bcl-2, Bcr-Abl, BRAF, BTK, BTLA, BTN2A1, CA-125, CAIX, CCR4, CD105/endoglin, CD109, CD123, CD155, CD16, CD160, CD19, CD20, CD200, CD200R, CD22, CD24, CD25, CD27, CD28, CD30, CD33, CD36, CD37, CD38, CD40, CD40L, CD47, CD48, CD52, CD70, CD79b, CD80, CD86, CD96, CDK4, CDK6, CDK9, CEA, CEACAM1, ChK1, ChK2, c-KIT, c-Met/HGFR, COX2, CSF-1R, CSF2, CTLA-4, CXCR2, CXCR4, DDR2, DLL3, DLL4, DNAM-1, DR5, EGFR, EpCAM, EPHA3, EphB4, ERK1, ERK2/p38 MAPK, FAK, FAP, FGF-2, FGFR1, FGFR2, FGFR3, FGFR4, Flt-3, Gal-9, GITR, GITRL, Glypican-3, HDAC1, HDAC2, HDAC3, HDAC4, HDAC5, HDAC7, HDAC8, HDAC9, HER2, HER3, HER4/ERBB4, HGF, HHLA2, HIF-1α, HSP27, HSP90, HVEM, ICOS, ICOS Ligand, IDO, IGF1R, IL-13, IL-6, JAK1, JAK2, JAK3, KRAS, LAG-3, LIGHT, MDM2, MEK1, MEK2, MMP-1, MMP-10, MMP-11, MMP-13, MMP-2, MMP-7, MMP-9, mTOR, Mucin 1, Myc, NF-κB, NKG2A, NRAS, NTRK1, NTRK2, NTRK3, OX40, OX40L, p53, PAF, PARP1, PARP2, PD1, PDGFR-α, PDGFR-β, PD-L1, PD-L2, PI3Kα, PI3Kβ, PI3Kγ, PI3Kδ, PIM1, PIM3, PSMA, PTEN, RAF-1, RANKL, RET, S100A4, SIRPα, SLAMF7, SMO, Src, STAT3, STEAP-1, Syk, TDO, TGFβ, Tie-2, TIGIT, TIM-3, TLR8, TMIGD2, TNF-α, Toll-like receptor 3, TRAIL, TRAILR1, TROP-2, VEGF, VEGF-C, VEGFR-1, VEGFR-2, VEGFR-3, VISTA, γ-secretase, or any combination thereof.

Other aspects of the present disclosure provide for kits for practicing any of the methods disclosed herein. In some embodiments, kits herein can include one or more lentiviral vectors and at least one container.

### BRIEF DESCRIPTION OF THE DRAWINGS

Embodiments of the present inventive concept are illustrated by way of example in which like reference numerals indicate similar elements and in which:

FIG. 1 depicts an image illustrating a schematic of the methods herein for mutagenesis screening in drug-sensitive cells using a lentivirus system.

FIGS. 2A-2C depict images illustrating human non-small cell lung cancer (NSCLC) cells infected with lentiviral particles containing a doxycycline-inducible RUVBL1 cDNA as the cargo DNA. FIG. 2A shows a representative image of uninduced (−doxycycline) NSCLC cells treated with RUVBL1/2 inhibitor. FIG. 2B shows a representative image of NSCLC cells having induced expression (+ doxycycline) of the RUVBL1 alleles treated with RUVBL1/2 inhibitor. FIG. 2C shows a graph depicting the enrichment of specific mutations in the RUVBL1 open reading frame as assessed by next generation sequencing of DNA harvested from cells shown in FIG. 2B.

FIGS. 3A-3C depict images illustrating human NSCLC cells infected with lentiviral particles containing a doxycycline-inducible EGFR cDNA as the cargo DNA. FIG. 3A shows a representative image of uninduced (−doxycycline) NSCLC cells treated with the EGFR inhibitor Gefitinib. FIG. 3B shows a representative image of NSCLC cells having doxycycline-induced expression of the EGFR alleles treated with Gefitinib. FIG. 3C shows a graph depicting the enrichment of specific mutations in the EGFR open reading frame as assessed by next generation sequencing of DNA harvested from cells shown in FIG. 3B.

FIGS. 4A-4C depict images illustrating human NSCLC cells infected with lentiviral particles containing RUVBL1 cDNA as the cargo DNA and either a wild-type lentiviral reverse transcriptase (WT-RT) or a M230I mutated lentiviral reverse transcriptase (“M-RT”). FIG. 4A shows a representative image of NSCLC cells with WT-RT having doxycycline-induced expression of the RUVBL1 alleles and treated with RUVBL1/2 inhibitor. FIG. 4B shows a representative image of NSCLC cells with M-RT having doxycycline-induced expression of the RUVBL1 alleles and treated with RUVBL1/2 inhibitor. FIG. 4C shows a graph depicting the quantification of surviving cell colonies from FIG. 4A and FIG. 4B.

FIGS. 5A-5D depict images illustrating human NSCLC cells infected with lentiviral particles containing EGFR cDNA as the cargo DNA and either a wild-type lentiviral reverse transcriptase (WT-RT) or a M230I mutated lentiviral reverse transcriptase (“M-RT”). FIG. 5A shows a representative image of NSCLC cells with WT-RT having doxycycline-induced expression of the EGFR alleles and treated with the EGFR inhibitor Osimertinib. FIG. 5B shows a representative image of NSCLC cells with M-RT having doxycycline-induced expression of the EGFR alleles and treated with Osimertinib. FIG. 5C shows a graph depicting the quantification of surviving cell colonies from FIG. 5A and FIG. 5B. FIG. 5D shows representative images of HCC827 cells (harboring the EGFR^{del746-750} mutation) expressing TET3G, transduced with lentiviral particles generated from pLVX-TRE3G-EGFR^{del746-750}-puro packaged with either M-RT or D-RT at an MOI of ˜0.3. Transduced cells were selected with puromycin, then 2 million cells were seeded in 15-cm dishes and selected with 100 nM Osimertinib or 1 μM Gefitinib in the presence of 1 mg/ml doxycycline. On appearance of resistant colonies staining with crystal violet was performed.

FIG. 6 depicts an image illustrating the nucleic acid substitution rate in cargo DNA delivered into cells by a lentivirus having a M-RT or by a lentivirus having a WT-RT.

FIGS. 7A-7C depict images illustrating use of LentiMutate with BCR-ABL1-identified deletions in BCR-ABL1 that conferred imatinib resistance. FIG. 7A shows a schematic of domain structure of the BCR-ABL1 fusion protein p210 (top image) and BCR-ABL1 structural variants (SVs) inferred by sequencing of BCR-ABL1 in imatinib resistant K562 cells after LentiMutate (bottom image). FIGS. 7B and 7C show graphs of viability-based dose-response curves following imatinib (FIG. 7B) or asciminib (FIG. 7C) treatment in K562 cells harboring doxycycline-inducible BCR-ABL1 variants (35INS, NTCD, SV8, and SV11) with (+dox) or without (−dox) expression of the BCR-ABL1 variant. Values shown are averages±standard error of the mean (SEM) of 2 biological replicates.

FIGS. 8A-8G depict images illustrating that deletions in BCR-ABL1 conferred resistance to imatinib and similar deletions can be generated in CML patients via alternative splicing. FIG. 8A shows a schematic of the experiment performed to create the cells used in FIGS. 8B-8D. FIG. 8B shows a representative immunoblot demonstrating loss of endogenous BCR-ABL1 and expression of exogenous BCR-ABL1. FIG. 8C shows a graph depicting that K562 cells lacking endogenous BCR-ABL1 required expression of the BCR-ABL1 mutants for their viability. Background subtracted absorbance from the MTS assay is shown. FIG. 8D shows a graph demonstrating that expression of BCR-ABL1 deletion mutants without endogenous BCR-ABL1 conferred resistance to imatinib. FIG. 8E shows a heatmap illustrating the percent of split reads supporting each novel isoform (rows) in 120 CML patient samples (columns). “Exon10-v1” denotes reads between ABL1 exon 10 and the novel v1 exon, “v1+v2+v3+v4” denotes the summation of split reads between exon 10 and any novel exon in each patient, “v1/2/3/4-exon11” denotes split reads between the novel exon and ABL1 exon 11 (summed for each patient). FIG. 8F shows a schematic of the various BCR-ABL1 isoforms identified by split reads. FIG. 8G shows a graph demonstrating expression of BCR-ABL1 lacking ABL1 exon 11 in K562 conferred resistance to imatinib. All values shown are averages±SEM of 2 biological replicates.

FIGS. 9A-9H depict images illustrating that LentiMutate identified mutations in WT KRAS or KRAS-G12C that confered resistance to AMG 510. FIGS. 9A and 9B show graphs of the results of LentiMutate on WT KRAS (FIG. 9A) and on KRAS-G12C (FIG. 9B) using 1 μM AMG 510 in the H358 cell line. Only substitutions enriched >3-fold are annotated, and values are the average of two biological replicates. S=synonymous mutation. FIGS. 9C-9F show structural images of the resistance mutations modeled using the KRAS-G12C:AMG 510 co-crystal structure (PDB 6OIM). AMG 510 is cyan, KRAS-G12C is white, oxygens are red, and nitrogens are blue. FIG. 9C V9 was normally buried in the hydrophobic core of the structure. In FIG. 9D, the V9D substitution was predicted to expel the side chain out towards the ligand binding pocket resulting in a steric clash. Red disks indicate significant van der Waals overlap between adjacent atoms. In FIG. 9E, Y96 normally participated in a network of hydrogen bonds including waters (black spheres) and hydrophobic contacts with the pyrodopyrimidine scaffold. In FIG. 9F, Y96H mutations resulted in loss of waters and hydrophobic contacts. FIGS. 9G and 9H show graphs demonstrating that expression of indicated KRAS point mutants identified by LentiMutate in H358 cells conferred resistance to AMG 510 (FIG. 9G) and MRTX849 (FIG. 9H). Values are averages±SEM of 2 biological replicates.

The drawing figures do not limit the present inventive concept to the specific embodiments disclosed and described herein. The drawings are not necessarily to scale, emphasis instead being placed on clearly illustrating principles of certain embodiments of the present inventive concept.

### DETAILED DESCRIPTION

The following detailed description references the accompanying drawings that illustrate various embodiments of the present inventive concept. The drawings and description are intended to describe aspects and embodiments of the present inventive concept in sufficient detail to enable those skilled in the art to practice the present inventive concept. Other components can be utilized, and changes can be made without departing from the scope of the present inventive concept. The following description is, therefore, not to be taken in a limiting sense. The scope of the present inventive concept is defined only by the appended claims, along with the full scope of equivalents to which such claims are entitled.

The present disclosure is based, in part, on the novel finding that transducing cells with lentiviral particles creates mutations upon integration into a target cell genome and those mutations may confer one or more phenotypic properties to the protein expressed by the heterologous nucleic acid sequence delivered to the target cell. Accordingly, the present disclosure provides for methods and compositions for the detection of and screening for mutations that convey phenotypic properties in a protein such as, for example, drug-resistance, and kits used in practicing the methods disclosed herein.

### I. Terminology

The phraseology and terminology employed herein are for the purpose of description and should not be regarded as limiting. For example, the use of a singular term, such as, “a” is not intended as limiting of the number of items. Also, the use of relational terms such as, but not limited to, “top,” “bottom,” “left,” “right,” “upper,” “lower,” “down,” “up,” and “side,” are used in the description for clarity in specific reference to the figures and are not intended to limit the scope of the present inventive concept or the appended claims.

Further, as the present inventive concept is susceptible to embodiments of many different forms, it is intended that the present disclosure be considered as an example of the principles of the present inventive concept and not intended to limit the present inventive concept to the specific embodiments shown and described. Any one of the features of the present inventive concept may be used separately or in combination with any other feature. References to the terms “embodiment,” “embodiments,” and/or the like in the description mean that the feature and/or features being referred to are included in, at least, one aspect of the description. Separate references to the terms “embodiment,” “embodiments,” and/or the like in the description do not necessarily refer to the same embodiment and are also not mutually exclusive unless so stated and/or except as will be readily apparent to those skilled in the art from the description. For example, a feature, structure, process, step, action, or the like described in one embodiment may also be included in other embodiments but is not necessarily included. Thus, the present inventive concept may include a variety of combinations and/or integrations of the embodiments described herein. Additionally, all aspects of the present disclosure, as described herein, are not essential for its practice. Likewise, other systems, methods, features, and advantages of the present inventive concept will be, or become, apparent to one with skill in the art upon examination of the figures and the description. It is intended that all such additional systems, methods, features, and advantages be included within this description, be within the scope of the present inventive concept, and be encompassed by the claims.

Any term of degree such as, but not limited to, “substantially” as used in the description and the appended claims, should be understood to include an exact, or a similar, but not exact configuration. For example, “a substantially planar surface” means having an exact planar surface or a similar, but not exact planar surface. Similarly, the terms “about” or “approximately,” as used in the description and the appended claims, should be understood to include the recited values or a value that is three times greater or one third of the recited values. For example, about 3 mm includes all values from 1 mm to 9 mm, and approximately 50 degrees includes all values from 16.6 degrees to 150 degrees. For example, they can refer to less than or equal to ±5%, such as less than or equal to ±2%, such as less than or equal to 1%, such as less than or equal to ±0.5%, such as less than or equal to ±0.2%, such as less than or equal to ±0.1%, such as less than or equal to ±0.05%.

The terms “comprising,” “including” and “having” are used interchangeably in this disclosure. The terms “comprising,” “including” and “having” mean to include, but not necessarily be limited to the things so described.

The terms “or” and “and/or,” as used herein, are to be interpreted as inclusive or meaning any one or any combination. Therefore, “A, B or C” or “A, B and/or C” mean any of the following: “A,” “B” or “C”; “A and B”; “A and C”; “B and C”; “A, B and C.” An exception to this definition will occur only when a combination of elements, functions, steps, or acts are in some way inherently mutually exclusive.

The term “biomolecule” as used herein refers to, but is not limited to, peptides, proteins, enzymes, antibodies, aptamers, nucleic acids, DNA, RNA siRNA, oligonucleotides, and small molecules. “Small molecules” as used herein can refer to chemicals, compounds, drugs, and the like.

The term “nucleic acid” or “polynucleotide” refers to deoxyribonucleic acids (DNA) or ribonucleic acids (RNA) and polymers thereof in either single- or double-stranded form. Unless specifically limited, the term encompasses nucleic acids containing known analogues of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions), alleles, orthologs, SNPs, and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); and Rossolini et al., Mol. Cell. Probes 8:91-98 (1994)).

The terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides, and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. A polypeptide includes a natural peptide, a recombinant peptide, or a combination thereof.

The term “encode” is used herein to refer to the capacity of a nucleic acid to serve as a template for transcription of RNA or the capacity of a nucleic acid to be translated to yield a polypeptide. Thus, a DNA sequence that is transcribed to yield an RNA is said to “encode” the RNA. If a nucleic acid sequence is transcribed to yield an RNA that is translated to yield a polypeptide, both the nucleic acid and the RNA are said to encode the polypeptide. “Transcription” as used herein includes reverse transcription, where appropriate.

As used herein, “operatively linked” or “operably linked” expression control sequences refers to a linkage in which the expression control sequence is contiguous with the gene of interest to control the gene of interest, as well as expression control sequences that act in trans or at a distance to control the gene of interest.

It should also be understood that, unless clearly indicated to the contrary, in any methods claimed herein that include more than one step or act, the order of the steps or acts of the method is not necessarily limited to the order in which the steps or acts of the method are recited.

### II. Compositions

Lentivirus is a genus of retroviruses that in nature give rise to slowly developing disease due to their ability to incorporate into a host genome. Modified lentiviral genomes are useful as viral vectors for the delivery of a gene to a host cell. The term “lentiviral vector” as used herein can refer to a vector including one or more “heterologous” (i.e., non-lentiviral) nucleic acid sequences. In some embodiments, lentiviral vectors herein may contain non-coding sequences of one or more proteins from a lentivirus. A “lentiviral transfer vector” for use herein may include a heterologous nucleic acid sequence, for example, to be transferred into a cell, and may further include, for example, one or more lentiviral genes, or portions thereof. A “lentiviral packaging vector” for use herein may include one or more genes encoding lentiviral proteins, or portions thereof. For example, a lentiviral envelope protein may include a gene encoding an envelope (Env) protein, or a portion thereof. In certain embodiments, host cells can be transfected with lentiviral vectors, and optionally additional vectors for expressing lentiviral packaging proteins (e.g., VSV-G, Rev, and Gag/Pol) to produce lentiviral particles in the culture medium. In some embodiments, lentiviral particles having a virus produced herein can in turn to be used to infect target cells to express one or more transgenes within the target cells. As used herein, a “transgene” refers to a gene that is transferred into a target cell, for example, using a lentiviral transfer vector of the present disclosure. In some embodiments, a transgene can be a gene encoding a target protein such as those described herein.

The present disclosure is based, in part, on the novel finding that when transducing cells with lentiviral particles that encompass a heterologous nucleic acid sequence, mutations can be created in the heterologous nucleic acid sequence upon integration into a target cell genome and those mutations can confer one or more phenotypic properties to the protein expressed by the mutated heterologous nucleic acid sequence that is delivered into the target cell. In some embodiments, the one or more phenotypic properties introduced in the protein expressed by the mutated heterologous nucleic acid sequence can be resistance to a biomolecule. As used herein, “resistance” can be a reduction in effectiveness of a biomolecule to exert the known effect it should have on to the protein. As an example, but not limited to, a protein having resistance to a biomolecule can demonstrate a reduction in effectiveness of a biomolecule to treat a disease or condition. In some embodiments, the one or more phenotypic properties introduced in the protein expressed by the mutated heterologous nucleic acid sequence can be resistance to a small molecule. In some embodiments, the one or more phenotypic properties introduced in the protein expressed by the mutated heterologous nucleic acid sequence can be resistance to a drug for the treatment of a disease. In some embodiments, the one or more phenotypic properties introduced in the protein expressed by the mutated heterologous nucleic acid sequence can be resistance to a drug for the treatment of cancer.
(a) Lentiviral Vectors

In certain embodiments, compositions, and methods of using compositions herein can include at least one viral vector. As used herein, the term “viral vector” can refer to a nucleic acid vector construct that includes at least one element of viral origin and has the capacity to be packaged into a viral vector particle and encodes at least an exogenous nucleic acid. The vector and/or particle can be utilized for the purpose of transferring any nucleic acids into cells either in vitro or in vivo. Numerous forms of viral vectors are known in the art. The term virion is used to refer to a single infective viral particle. “Viral vector”, “viral vector particle” and “viral particle” also refer to a complete virus particle with its DNA or RNA core and protein coat as it exists outside the cell.

In certain embodiments, a viral vector disclosed herein can be a lentiviral vector. Examples of lentiviruses include, but are not limited to, human lentiviruses such as HIV (in particular HIV-1 or HIV-2), simian immunodeficiency virus (SIV), equine infectious anemia virus (EIAV), feline immunodeficiency virus (FIV), Caprine Arthritis Encephalitis Virus (CAEV), visna and progressive pneumonia viruses of sheep, bovine immunodeficiency virus (BIV), and the like.

The lentiviral genome of the lentiviral vectors disclosed herein includes three genes found in retroviruses: gag, pol and env, which are flanked by two long terminal repeat (LTR) sequences. The gag gene encodes the internal structural proteins, such as matrix, capsid and nucleocapsid proteins; the pol gene encodes the RNA-directed DNA polymerase (reverse transcriptase (RT)), a protease and an integrase; and the env gene encodes viral envelope glycoproteins. The 5′ and 3′ LTR's serve to promote transcription and polyadenylation of the virion RNA's. Lentiviral transfer vectors for use herein can include elements suitable for enabling transfer of heterologous nucleic acids present in the lentiviral transfer vector into a cell (e.g., a cell transfected with the lentiviral transfer vector, and optionally one or more additional vectors, such as packaging vectors). In some embodiments, heterologous nucleic acids present in the lentiviral transfer vector can include a heterologous nucleic acid sequence, such as, for example, a sequence encoding a transgene.

In some embodiments, lentiviral vectors disclosed herein may comprise a 5′ lentiviral long terminal repeat (5′ LTR) and a 3′ lentiviral long terminal repeat (3′ LTR). The 5′ LTR and/or 3′ LTR can be the native 5′ LTR and native 3′ LTR of a lentiviral genome. Alternatively, either one may be modified, e.g., including deletions, insertions, and/or mutations relative to the native sequences. In some examples, the 3′-LTR may further comprise a polyadenylation (poly A) enhancer signal sequence, which is located upstream of the cleavage/polyadenylation (polyA) site (e.g., AAUAAA) and function to increase the polyA site efficiency and thus polyadenylation efficiency. In some embodiments, a polyadenylation enhancer signal sequence can include upstream sequence element (USE) from a suitable viral gene, for example, simian virus 40 (SV40) late gene. Inclusion of such a polyA enhancer signal sequence may facilitate transcription termination and reduce read-through of vector transcript and improving packaging efficiency, which would lead to increased viral titer.

In some instances, lentiviral vectors disclosed herein can be a self-inactivating (SIN) vector, which may contain a deletion in the 3′ long terminal repeat region (LTR). In some examples, lentiviral vectors may contain a deletion within the viral promoter.

In addition to the LTRs described herein, lentiviral vectors disclosed herein can also have one or more components necessary for the basic functionality of the viral vector, for example, capable of being replicated, packed into viral particles, and/or capable of drive expression of genes of interest carried thereby in host cells. Such essential elements for constructing lentiviral vectors are well known to those skilled in the art.

In some embodiments, lentiviral vectors described herein may comprise one or more of the following components: (i) a psi (p) packaging signal; (ii) a rev response element (RRE); (iii) a gag element; (iv) an env splice acceptor sequence; (v) one or more copies of a heterologous polyA signal sequence downstream from the 3′ LTR; (vi) one or more chromatin insulator elements; (vii) a central polypurine tract (cPPT); and (viii) a post-transcriptional regulatory element (PRE).

A psi (ψ) packaging signal, also known as an encapsidation sequence, can regulate the packaging of retroviral RNA into viral capsids during replication. It is typically placed downstream of 5′ long terminal repeat in a lentiviral transfer vector to effectively package and deliver transgene carried by the lentiviral transfer vector.

A rev response element (RRE) can be a domain located in the env region. A RRE may have up to 360 nucleotides long within the ‘env gene’. Rev protein can bind to the RRE to regulate the expression of viral genes. The Rev/RRE system facilitates nuclear export of mRNAs.

A gag (group-specific antigen) element encodes for the structural proteins (or a portion thereof) of a retrovirus, i.e., matrix, capsid and nucleocapsid components. In some embodiments, lentiviral vectors described herein may contain a gag fragment that is the 5′ fragment of a gag gene. Such a fragment may contain 250-650 bps (e.g., about 360 bps or 600 bps). Containing such a short gag fragment may enhance viral titer of retroviral vectors carrying a large gene of interest (for example, a globin gene). In some embodiments, lentiviral vectors described herein may be free of any gag fragment.

An env splice acceptor sequence is a nucleotide sequence near the 3′ end of the pol coding region in a lentiviral genome. The splice acceptor sequence regulates the splicing of transcripts. It also enables the expression of the env coding region. In some instances, lentiviral vectors described herein may comprise one or more heterologous polyA signaling sites, which may be located downstream from the 3′ LTR. Such heterologous polyA signaling sites may not be of a viral origin (e.g., from a non-viral gene such as a β-globin gene). Alternatively, the heterologous polyA signaling sites may be derived from a viral gene which is from a different viral species as the retroviral vector that contains the heterologous polyA signaling sites. Inclusion of such heterologous polyA signaling sites may enhance polyadenylation efficiency, thereby further reducing read-through of vector transcript and improving packaging efficiency, which would lead to increased viral titer.

In some embodiments, lentiviral vectors described herein may include one or more chromatin insulator elements. Chromatin insulators are promoter or enhancer sequences that resist heterochromatin formation. In some embodiments, a chromatin insulator can be a fragment of about 1 kb in length that blocks transcriptional activation by enhancers. It may function as barrier elements, as described herein to, inter alia, prevent the spread of heterochromatin and silencing of genes, reduce chromatin position effects and have enhancer blocking activity. These properties are desirable for consistent predictable expression and safe transgene delivery with randomly integrating vectors. Insulated vectors have reduced chromatin position effects and provide consistent, and therefore improved overall expression. In some examples, the one or more chromatin insulator elements in the retroviral vector described herein may be chicken hypersensitive site-4 elements (cHS4), which is a chromatin insulator from the chicken β-globin locus control region. In some instances, one or more full-length chromatin insulators (about 1.2 kb) of hypersensitive site-4 (cHS4) from the chicken β-globin locus can be inserted in the 3′LTR to allow its duplication into the 5′LTR in gamma-retrovirus and lentivirus vectors. In other instances, a truncated cHS4 fragment comprising a ˜250-bp core may be used in the retroviral vector described herein. Such a core fragment may be combined with a 3′˜400-bp fragment from the cHS4 element. In some examples, a functional reduced-length insulator of about 650 base pairs, including the core sequence and the 3′-fragment, can be used in constructing the retroviral vector described herein. Non-limiting examples of other chromatin insulators can include Arsl (derived from the sea urchin arylsulfatase gene locus), sns5 (derived from the sea urchin H2A early histone gene), Ankyrin-1 gene promoter element, and Drosophila gypsy element.

A central polypurine tract (cPPT) directs penetration of viral particles through the nuclear membrane. In lentiviral replication, it can function as a primer for synthesis of plus-strand DNA. It has been shown to increase the transduction efficiency and transgene expression when incorporated into lentiviral vectors.

A post-transcriptional regulatory element (PRE) is a sequence that, when transcribed, enhances the expression of a transgene in a viral vector. It has been shown to increase the transduction efficiency and transgene expression when incorporated into retroviral vectors. In some embodiments, the PRE used in the lentiviral vectors herein can be a PRE from a Hepatitis B virus (HPRE) or a PRE from a Woodchuck Hepatitis virus (WPRE). In some embodiments, there can be more than one PRE in the retroviral vector, and the more than one PRE can be HPRE, WPRE, or a mixture thereof. In some embodiments, lentiviral vectors herein do not include a PRE.

In some embodiments, lentiviral vectors herein may include one or more of the following: a promoter (e.g., a CMV, RSV, or EF1a promoter) driving expression of one or more viral sequences, long terminal repeat (LTR) regions (e.g., an R region or an U5 region), a primer binding site (PBS), a packaging signal (psi) (e.g., a packaging signal including a major splice donor site (SD)), a partial gag sequence (e.g., as described herein), a partial env sequence, a Rev-response element (RRE), additional partial env sequence, optionally including a splice acceptor site (e.g., an SA7 splice acceptor), a partial pol sequence including a central polypurine tract (cPPT) (e.g., a cPPT comprising a splice acceptor site, e.g., SA1), a subgenomic promoter (e.g., P-EF1a), a nucleic acid (e.g., a heterologous nucleic acid including a gene encoding a transgene of interest), a post-transcriptional regulatory element (e.g., a WPRE or HPRE, optionally including an X protein mutation), a polyA sequence (e.g., an SV40 polyA tail), a selectable marker (e.g., a kanamycin resistance gene (nptll), ampicillin resistance gene, or a chloramphenicol resistance gene), and an origin of replication (e.g., a pUC origin of replication, an SV40 origin of replication, or an f1 origin of replication). In some embodiments, lentiviral vectors herein may also include additional sequences (e.g., vector backbone sequences), such as those well known in the art. In some embodiments, lentiviral vectors herein may include or incorporate sequences from the vectors described herein (e.g., pNOX, pCINS, and/or pNLV). In some embodiments, lentiviral vectors herein may include or incorporate vector backbone sequences, or portions thereof, from vectors described herein (e.g., pNOX, pCINS, and/or pNLV).

In some embodiments, lentiviral transfer vectors herein may also include elements suitable for driving expression of protein in a target cell. In certain instances, a Kozak sequence can be positioned upstream of the protein open reading frame. In some examples, lentiviral transfer vectors may include a promoter (e.g., a CMV, RSV, or EF1a promoter) that controls the expression of the heterologous nucleic acid sequence. Other promoters suitable for use in the lentiviral transfer vectors include, for example, constitutive promoters or tissue/cell type-specific promoters. In some embodiments, a lentiviral transfer vector includes a means of selectively marking a gene product (e.g., a polypeptide or RNA) encoded by at least a portion of the heterologous nucleic acid (e.g., a gene product of interest). For example, the lentiviral transfer vector may include a marker gene. In some embodiments, a marker gene can be a gene encoding for a selectable marker. In some embodiments, a selectable marker can a fluorescent protein (e.g., GFP, YFP, RFP, dsRed, mCherry, or any derivative thereof). In some embodiments, a selectable marker can an antibiotic resistance gene (e.g., hygromycin resistance gene (hph), puromycin resistance gene (PuroR), blasticidin S-resistance gene (bsr), tetracycline resistance genes (tet), neomycin resistance gene (neo), Zeocin resistance gene (Sh ble), or any derivative thereof). In some examples, a marker gene may be expressed independently of the gene product of interest. In some examples, a marker gene may be co-expressed with the gene product of interest. In some examples, a marker gene may be under the control of the same or different promoter as the gene product of interest. In some examples, a marker gene may be fused to the gene product of interest. The elements of the lentiviral transfer vectors disclosed herein are, in general, in operable association with one another, to enable the transfer vectors to participate in the formation of a lentivirus in a transfected cell, together with one or more packaging vectors.

In some embodiments, lentiviral transfer vectors herein may have a native promoter for the transgene. In some embodiments, a native promoter may be used when it is desired that expression of the gene should mimic the native expression. In some embodiments, a native promoter may be used when expression of the gene must be regulated temporally or developmentally, or in a tissue-specific manner, or in response to specific transcriptional stimuli. In some embodiments, other native expression control elements, such as enhancer elements, polyadenylation sites or Kozak consensus sequences may also be used to mimic the native expression. In some embodiments, the transgene product or other desirable product to be expressed is operably linked to a tissue-specific promoter. According to some embodiments of the present disclosure, a cell type specific promoter is specific for cell types found in the brain (e.g., neurons, glial cells), liver (e.g., hepatocytes), pancreas, skeletal muscle (e.g., myocytes), immune system (e.g., T cells, B cells, macrophages), heart (e.g., cardiac myocytes), retina, skin (e.g., keratinocytes), bone (e.g., osteoblasts or osteoclasts), and the like. One of skill in the art can appreciate that a tissue-specific promoter can also be used in cells sharing the targeted tissue origin.

In some embodiments, lentiviral transfer vectors herein may have a promoter allowing for inducible expression of the sequence of interest. As used herein, the term “inducible promoter” refers to a regulatory element (e.g., a promoter, promoter/enhancer, or portion thereof) whose transcriptional activity may be regulated by exposing a cell or tissue comprising a nucleic acid sequence operably linked to the promoter to a treatment or condition that alters the transcriptional activity of the promoter, resulting in increased transcription of the nucleic acid sequence. As used herein, the term “inducible promoter” also includes repressible promoters, i.e., promoters whose transcriptional activity may be regulated by exposing a cell or tissue comprising a nucleic acid sequence operably linked to the promoter to a treatment or condition that alters the transcriptional activity of the promoter, resulting in decreased transcription of the nucleic acid sequence. A number of systems for inducible expression are known in the art, including the tetracycline responsive system, the lac operator-repressor system, as well as promoters responsive to a variety of environmental or physiological changes, including heat shock, metal ions, such as metallothionein promoter, interferons, hypoxia, steroids, such as progesterone or glucocorticoid receptor promoter, radiation, such as VEGF promoter. Inducible promoters suitable for use herein can be grouped as chemically-regulated promoters, and/or physically-regulated promoters. Chemically-regulated promoters can include, not are not limited to, alcohol-regulated promoters (e.g., alcohol dehydrogenase I (alcA) gene promoter), tetracycline-regulated promoters (e.g., tetracycline-responsive promoter), steroid-regulated promoter (e.g., rat glucocorticoid receptor (GR)-based promoter, human estrogen receptor (ER)-based promoter, moth ecdysone receptor-based promoter, and the promoters based on the steroid/retinoid/thyroid receptor superfamily), metal-regulated promoters (e.g., metallothionein gene-based promoters), and pathogenesis-related promoters (e.g., Arabidopsis and maize pathogen-related (PR) protein-based promoters). Typical physically-regulated promoters can include, but are not limited to, temperature-regulated promoters (e.g., heat shock promoters), and light-regulated promoters (e.g., soybean SSU promoter).

In some embodiments, lentiviral vectors herein may have one or more tetracycline-regulated promoters. In some embodiments, lentiviral vectors herein can have a tetracycline-controlled transactivator and/or reverse tetracycline-controlled transactivator, in some embodiments, lentiviral transfer vectors herein can have operator sequences of the tet operon to which the tetracycline-controlled transactivator and/or reverse tetracycline-controlled transactivator specifically bind

In some embodiments, lentiviral transfer vectors herein can have at least one tetracycline-regulated promoter can be upstream of a heterologous nucleic acid sequence encoding for a transgene of interest. In some embodiments, transgene expression from lentiviral transfer vectors herein having at least one tetracycline-regulated promoter can be induced in the presence of tetracycline and/or doxycycline. In some examples, transgene expression from lentiviral transfer vectors herein can be induced in the presence of tetracycline and/or doxycycline in a dose-dependent manner. In some examples, a dose of tetracycline and/or doxycycline needed to induce transgene expression from a lentiviral vector herein can be dependent on cell type. In some examples, a dose of tetracycline and/or doxycycline needed to induce transgene expression from a lentiviral vector herein can be between about 0.1 μg/ml to about 2.0 μg/ml (e.g., about 0.1 μg/ml, about 0.5 μg/ml, about 1.0 μg/ml, about 1.5 μg/ml, about 2.0 μg/ml).

In some embodiments, lentiviral transfer vectors herein can have a combination of two or more promoters disclosed herein to obtain the desired expression of the gene of interest. The artisan of ordinary skill will be able to select a promoter based on the desired expression pattern of the gene in the organism or the target cell of interest.

In certain embodiments, “lentiviral packaging vectors” for use herein may include one or more genes encoding lentiviral proteins, or portions thereof. In some embodiments, a lentiviral packaging vector herein can include a gene encoding an envelope (env) protein, a truncation of an env protein, a fragment of an env protein, a variant of an env protein, an env protein having one or more mutations in its amino acid sequence, or any combination thereof. In some embodiments, a lentiviral packaging vector herein can encode for an envelope (env) protein and a viral glycoprotein (e.g., VSV-G). Encoding a viral glycoprotein in addition to env can provide the vector particles with a receptor binding protein.

In some embodiments, a lentiviral packaging vector herein can include a gene encoding a gag protein, a truncation of a gag protein, a fragment of a gag protein, a variant of a gag protein, a gag protein having one or more mutations in its amino acid sequence, or any combination thereof. In some embodiments, a lentiviral packaging vector herein can include a gene encoding a pol protein, a truncation of a pol protein, a fragment of a pol protein, a variant of a pol protein, a pol protein having one or more mutations in its amino acid sequence, or any combination thereof. In some embodiments, a lentiviral packaging vector herein can encode for gag, pol and/or one or more regulatory/accessory proteins. In some embodiments, a lentiviral packaging vector herein can encode for gag, pol and one or more regulatory/accessory proteins under the control of one promoter. In some embodiments, a lentiviral packaging vector herein can encode for gag, pol and one or more regulatory/accessory proteins under the control of a CMV promoter.

In some embodiments, a lentiviral packaging vector herein can have a gene encoding for a pol protein, wherein the pol protein can include a reverse transcriptase (RT) of the lentivirus, protease (PRO) of the lentivirus, integrase (IN) of the lentivirus, Rnase H (RN) of the lentivirus, or any combination thereof. In some embodiments, a lentiviral packaging vector herein can have a gene encoding for a pol protein, wherein the pol protein can include PRO, a truncation of PRO, a fragment of PRO, a variant of PRO, a PRO protein having one or more mutations in its amino acid sequence, or any combination thereof. In some embodiments, a lentiviral packaging vector herein can have a gene encoding for a pol protein, wherein the pol protein can include IM, a truncation of IN, a fragment of IN, a variant of IN, an IN protein having one or more mutations in its amino acid sequence, or any combination thereof. In some embodiments, a lentiviral packaging vector herein can have a gene encoding for a pol protein, wherein the pol protein can include RN, a truncation of RN, a fragment of RN, a variant of RN, a RN protein having one or more mutations in its amino acid sequence, or any combination thereof. In some embodiments, a lentiviral packaging vector herein can have a gene encoding for a pol protein, wherein the pol protein can include RT, a truncation of RT, a fragment of RT, a variant of RT, a RT protein having one or more mutations in its amino acid sequence, or any combination thereof. In some embodiments, a lentiviral packaging vector herein can have a gene encoding for a pol protein, wherein the pol protein can include a RT having an amino acid sequence that is about 80% to 100% identical to SEQ ID NO: 1 (e.g., about 80%, 85%, 90%, 95%, 99%, 100%). The amino acid sequence of SEQ ID NO: 1 is provided in Table 1 below.

Like all retroviruses, the viral genome herein is first reverse transcribed by RT into cDNA, which integrates into the host cell genome after a cellular DNA polymerase synthesizes the corresponding anti-sense strand. In some embodiments, a lentiviral packaging vector herein can have a gene encoding for a pol protein, wherein the pol protein can include a RT protein having one or more mutations in its amino acid sequence. In some embodiments, a lentiviral packaging vector herein can have a gene encoding for a pol protein, wherein the pol protein can include a RT protein having one or more mutations in its amino acid sequence synthetically produced via directed-protein evolution. Directed-protein evolution is a protein engineering strategy used in the field for improving one or more desired biological functions of proteins by repeated rounds of mutation and selection. In accordance with embodiments herein, a RT protein having one or more mutations in its amino acid sequence herein can result from directed-protein evolution to “improve” (i.e., increase) the rate at which a nucleic acid of the viral genome is improperly reverse transcribed (i.e., “substituted”) into cDNA.

In some embodiments, a lentiviral packaging vector herein can have a gene encoding for a pol protein, wherein the pol protein can include a RT protein having one or more mutations in its amino acid sequence associated with resistance to non-nucleoside reverse transcriptase inhibitors (NNRTIs). In some embodiments, a lentiviral packaging vector herein can have a gene encoding for a pol protein, wherein the pol protein can encompass a RT protein having one or more mutations of the following mutations in its amino acid sequence: V901, A98G, L1001, K101E, K101P, K101Q, K103H, K103N, K103S, K103T, V106A, V1061, V106M, V1081, E138G, E138K, E138Q, V179D, V179E, V179F, V179G, V1791, Y181C, Y1811, Y181V, Y188C, Y188H, Y188L, V1891, G190A, G190C, G190E, G190Q, G190S, H221Y, P225H, F227C, F227L, M230I, M230L, P236L, K238N, K238T, Y318F, and Y501W. In some embodiments, a lentiviral packaging vector herein may comprise a gene encoding for a pol protein, wherein the pol protein can encompass a RT protein having a M230I mutation in its amino acid sequence with reference to SEQ ID NO: 1. In some embodiments, a lentiviral packaging vector herein may comprise a gene encoding for a pol protein, wherein the pol protein can encompass a RT protein having a sequence as set forth in SEQ ID NO: 2 provided in Table 1 below. In some embodiments, a lentiviral packaging vector herein can have a gene encoding for a pol protein, wherein the pol protein can include a RT having an amino acid sequence that is about 80% to 100% identical to SEQ ID NO: 2 (e.g., about 80%, 85%, 90%, 95%, 99%, 100%). In some embodiments, a lentiviral packaging vector herein may comprise a gene encoding for a pol protein, wherein the pol protein comprises a RT protein comprising a M230I and Y501W mutation (D-RT) in its amino acid sequence with reference to SEQ ID NO: 1. In some embodiments, the reverse transcriptase may comprise an amino acid sequence as set forth in SEQ ID NO: 14, or a sequence at least about 80% identical thereto. The amino acid sequence of SEQ ID NO: 14 is provided in Table 1 below. In some embodiments, a lentiviral packaging vector herein can have a gene encoding for a pol protein, wherein the pol protein can include a RT having an amino acid sequence that is about 80% to 100% identical to SEQ ID NO: 14 e.g. about 80%, 85%, 90%, 95%, 99%, 100%).

TABLE 1

| | Amino Acid Sequence | SEQ ID NO |
|---|---|---|
| WT-RT | PISPIETVPVKLKPGMDGPKVKQWPLTEEKIKALVEICTEMEKEGKIS | 1 |
| | KIGPENPYNTPVFAIKKKDSTKWRKLVDFRELNKRTQDEWEVQLGIPH | |
| | PAGLKKKKSVTVLDVGDAYFSVPLDEDERKYTAFTIPSINNETPGIRY | |
| | QYNVLPQGWKGSPAIFQSSMTKILEPFRKQNPDIVIYQYMDDLYVGSD | |
| | LEIGQHRTKIEELRQHLLRWGLTTPDKKHQKEPPELWMGYELHPDKWT | |
| | VQPIVLPEKDSWTVNDIQKLVGKLNWASQIYPGIKVRQLCKLLRGTKA | |
| | LTEVIPLTEEAELELAENREILKEPVHGVYYDPSKDLIAEIQKQGQGQ | |
| | WTYQIYQEPFKNLKTGKYARMRGAHTNDVKQLTEAVQKITTESIVIWG | |
| | KTPKFKLPIQKETWETWWTEYWQATWIPEWEFVNTPPLVKLWYQLEKE | |
| | PIVGAETFYVDGAANRETKLGKAGYVTNRGRQKVVTLTDTTNQKTELQ | |
| | AIYLALQDSGLEVNIVTDSQYALGIIQAQPDQSESELVNQIIEQLIKK | |
| | EKVYLAWVPAHKGIGGNEQVDKLVSAGIRKVL | |
| RT- | PISPIETVPVKLKPGMDGPKVKQWPLTEEKIKALVEICTEMEKEGKIS | 2 |
| M2301 | KIGPENPYNTPVFAIKKKDSTKWRKLVDFRELNKRTQDFWEVQLGIPH | |
| | PAGLKKKKSVTVLDVGDAYFSVPLDEDERKYTAFTIPSINNETPGIRY | |
| | QYNVLPQGWKGSPAIFQSSMTKILEPFRKQNPDIVIYQYMDDLYVGSD | |
| | LEIGQHRTKIEELRQHLLRWGLTTPDKKHQKEPPFLWIGYELHPDKWT | |
| | VQPIVLPEKDSWTVNDIQKLVGKLNWASQIYPGIKVRQLCKLLRGTKA | |
| | LTEVIPLTEEAELELAENREILKEPVHGVYYDPSKDLIAEIQKQGQGQ | |
| | WTYQIYQEPFKNLKTGKYARMRGAHTNDVKQLTEAVQKITTESIVIWG | |
| | KTPKFKLPIQKETWETWWTEYWQATWIPEWEFVNTPPLVKLWYQLEKE | |
| | PIVGAETFYVDGAANRETKLGKAGYVTNRGRQKVVTLTDTTNQKTELQ | |
| | AIYLALQDSGLEVNIVTDSQYALGIIQAQPDQSESELVNQIIEQLIKK | |
| | EKVYLAWVPAHKGIGGNEQVDKLVSAGIRKVL | |
| D-RT- | PISPIETVPVKLKPGMDGPKVKQWPLTEEKIKALVEICTEMEKEGKIS | 14 |
| M2301, | KIGPENPYNTPVFAIKKKDSTKWRKLVDFRELNKRTQDFWEVQLGIPH | |
| Y501W | PAGLKKKKSVTVLDVGDAYFSVPLDEDERKYTAFTIPSINNETPGIRY | |
| | QYNVLPQGWKGSPAIFQSSMTKILEPFRKQNPDIVIYQYMDDLYVGSD | |
| | LEIGQHRTKIEELRQHLLRWGLTTPDKKHQKEPPELWIGYELHPDKWT | |
| | VQPIVLPEKDSWTVNDIQKLVGKLNWASQIYPGIKVRQLCKLLRGTKA | |
| | LTEVIPLTEEAELELAENREILKEPVHGVYYDPSKDLIAEIQKQGQGQ | |
| | WTYQIYQEPFKNLKTGKYARMRGAHTNDVKQLTEAVQKITTESIVIWG | |
| | KTPKFKLPIQKETWETWWTEYWQATWIPEWEFVNTPPLVKLWYQLEKE | |
| | PIVGAETFYVDGAANRETKLGKAGYVTNRGRQKVVTLTDTTNQKTELQ | |
| | AIYLALQDSGLEVNIVTDSQWALGIIQAQPDQSESELVNQIIEQLIKK | |
| | EKVYLAWVPAHKGIGGNEQVDKLVSAGIRKVL | |

In some embodiments, a lentiviral packaging vector herein can have a gene encoding for a pol protein that can encompass a RT protein having at least one mutation in its amino acid sequence, wherein the at least one mutation modifies the rate at which a nucleic acid of the viral genome is improperly reverse transcribed into cDNA compared to a RT protein not having any mutations in its amino acid sequence. In some embodiments, a RT protein having at least one mutation in its amino acid sequence herein, can modify (e.g., increase, decrease) the rate of substitutions, indels, point mutations, inversions, and the like during reverse transcription of the viral genome into cDNA compared to a RT protein not having any mutations in its amino acid sequence.

In some embodiments, a lentiviral packaging vector herein can have a gene encoding for a pol protein that can encompass a RT protein having at least one mutation in its amino acid sequence, wherein the at least one mutation increases the rate at which one or more indels are reverse transcribed into cDNA compared to a RT protein not having any mutations in its amino acid sequence. As used herein, an “indel” refers to an insertion or deletion of bases in the genome of an organism. In some embodiments, a RT protein having at least one mutation in its amino acid sequence herein can have an increased rate at which one or more indels are reverse transcribed into cDNA wherein an indel may be between about 1 base pair in length to about 10 base pairs in length (e.g., about 1, 2, 3, 4, 5, 6, 7, 8, 9, 10 base pairs). In some embodiments, a RT protein having at least one mutation in its amino acid sequence herein can have an increased rate at which one or more microindels are reverse transcribed into cDNA. In some embodiments, a RT protein having at least one mutation in its amino acid sequence herein can have an increased rate at which one or more microindels are reverse transcribed into cDNA wherein a microindel may be between about 1 nucleotide to about 50 nucleotides (e.g., about 1, 5, 10, 15, 20, 25, 30, 35, 40, 45, 50 nucleotides).

In some embodiments, a lentiviral packaging vector herein can have a gene encoding for a pol protein that can encompass a RT protein having at least one mutation in its amino acid sequence, wherein the at least one mutation increases the substitution rate when reverse transcribing the viral genome into cDNA compared to a RT protein not having any mutations in its amino acid sequence. In some examples, a mutated RT can have between about a 1-fold to about a 6-fold increase in substitution rate compared to an unmutated RT. In some examples, a mutated RT herein, when compared to an unmutated RT, can increase the rate of improperly substituting a C for a G, an A for a C, a T for a G, a G for a C, an A for a T, a T for an A, a G for a T, a T for a C, an A for a G, a C for an A, a C for a T, a G for an A, or any combination thereof when reverse transcribing the viral genome into cDNA. In some examples, a mutated RT herein, when compared to an unmutated RT, can increase the rate of improperly substituting for a C, a C for a T, a G for an A, or any combination thereof when reverse transcribing the viral genome into cDNA.

In some examples, a RT herein having a M230I mutation in its amino acid sequence or the double mutant having a M230I and Y501W mutation (D-RT) has an increased rate at which a nucleic acid of the viral genome is improperly reverse transcribed (i.e., “substituted”) into cDNA compared to a RT protein not having any mutations in its amino acid sequence. In some examples, a RT herein having a M230I mutation in its amino acid sequence or a D-RT has between about a 1-fold to about a 6-fold increase in substitution rate compared to a RT protein not having any mutations in its amino acid sequence. In some examples, a RT herein having a M230I mutation in its amino acid sequence or the D-RT has an increased rate of improperly substituting a C for a G, an A for a C, a T for a G, a G for a C, an A for a T, a T for an A, a G for a T, a T for a C, an A for a G, a C for an A, a C for a T, a G for an A, or any combination thereof when reverse transcribing the viral genome into cDNA compared to a RT protein not having any mutations in its amino acid sequence. In some examples, a RT herein having T for a C, a C for a T, a G for an A, or any combination thereof when reverse transcribing the viral genome into cDNA compared to a RT protein not having any mutations in its amino acid sequence.

Lentiviral vectors described herein may further have one or more additional functional elements as known in the art to address safety concerns and/or to improve vector functions, such as packaging efficiency and/or viral titer.

Lentiviral vectors described herein can be prepared by conventional recombinant technology. In some instances, lentiviral vectors described herein can be prepared via the conventional cloning technology. Molecular Cloning: A Laboratory Manual, Green, M. R. and Sambrook J., New York: Cold Spring Harbor Laboratory Press, 2012; Gibson, D. G., et al., Nature Methods 6(5):343-345 (2009), the teachings of which relating to molecular cloning are herein incorporated by reference. Alternatively, lentiviral vectors described herein can be prepared by a gene editing method (e.g., by CRISPR).
(b) Lentiviral Particles

In certain embodiments, any of the lentiviral vectors as disclosed herein can be introduced into suitable host cells permissive for production of lentiviral particles. Examples include, but are not limited to, 293T cells, 293FT cells, COS cells, L cells, 3T3 cells, and Chinese hamster ovary (CHO) cells. In some instances, the lentiviral vectors lack one or more lentiviral packaging proteins (e.g., those noted above). Such lentiviral vectors can be co-transfected with one or more additional vectors (e.g., any of the lentiviral packaging vectors disclosed herein) capable of expressing the lentiviral packaging proteins. For example, a lentiviral vector carrying a gene of interest may be co-transfected with three additional vectors, each being designed for expressing VSV-G protein, Rev protein, and gag/pol proteins.

Alternatively, lentiviral packaging cells can be used as the host cell. Such cells stably express lentiviral proteins essential for viral particle packaging. Any of the lentiviral vectors can be introduced into lentiviral packaging cells in the absence of other vectors for expressing packaging proteins. In some embodiments, a host cell can stably express one or more of the proteins similar to that encoded in the lentiviral packaging vectors disclosed herein. In some examples, a host cell can stably express a RT protein having at least one mutation in its amino acid sequence as disclosed herein.

Methods for transfecting viral vectors into host cells are well known in the art. Some transfection approaches are chemical, e.g., using liposomes or calcium phosphate. Others may be non-chemical, e.g., electroporation or optical transfection. In some examples, the methods disclosed herein may involve the use of polyethylenimine (PEI) for transfection.

To transfect suitable host cells with any of the lentiviral vectors disclosed herein, the cells can be seeded in a suitable container. Suitable containers include petri dishes, flasks, vials, multi-tray systems (Cell Factories, Cell Stacks), or similar containers suitable for cell culture. Many varieties are known in the art and are commercially available, for example Cell BIND™ plates and flasks from Corning™. When needed, the surface of the container can be pretreating with chemicals such as poly-L-lysine to increase cell adherence. Alternatively, the surface of the container may not be pretreated.

In some embodiments, the methods disclosed herein may use Cell Bind 10 layer cell stacks for cell growth and transfection. Using this type of containers can decrease the total number of stacks needed for manufacturing, and also eliminates the need to Poly-L-Lysine treat the stacks. This reduces the total number of open manipulations performed during the aseptic manufacturing procedure and eliminates the use of an unnecessary solution during manufacture.

In some embodiments, the host cells used herein can be cultured in the container for a suitable period in a suitable medium (complete medium) to allow growth of the cells to appropriate confluency and conditions for transfection. Complete medium is a term known in the art as referring to a medium for an in vitro culture that contains supplemental nutrients as well as basic nutrients to support cell growth requirements. For manufacturing purposes (for producing a large quantity of lentiviral particles), suspension cell culture may be used. Medium selection is often dependent on the host cells used. Culture media are widely available and known in the art, but will contain a carbon source, water, various salts, amino acids, and nitrogen, along with other nutrients or growth factors specifically tailored to the host cells. Exemplary culture media include Dulbecco's Modified Eagle's Medium (DMEM). Additionally, culture medium may be modified to suit the needs of the host cells used in the methods described herein, for example by the addition of other components such as fetal bovine serum (FBS), sodium-pyruvate, benzonase, magnesium chloride, chloroquine, a transfection agent or compound, or a combination thereof. In some examples, the culture medium used herein can be DMEM, which may be supplemented with FBS (e.g., 8 to 12% such as 10%), sodium-pyruvate (e.g., 0.5 to 3% such as 1%) or a combination thereof.

When the host cells are in condition for transfection, a mixture containing a lentiviral vector (e.g., those disclosed herein), the additional vectors if any, transfection agent, and the culture medium can be prepared and incubated for a suitable period. A transfection agent is a substance that facilitates entry of the DNAs into the host cells. Many transfection agents are known in the art and widely available. Examples include calcium phosphate, highly branched organic compounds, cataionic polymers (such as polyethylenimine), and liposomes.

The mixture containing the DNAs and transfection agent can then be added to the cell culture to allow for delivery of the lentiviral vector and any additional vectors into the host cells. In some embodiments, the method disclosed herein involves incubating the transfection mixture with host cells growing in suspension. After a suitable period, the whole content, including the host cells and the transfection mixture, may be placed in a culture containing having multiple stacks (e.g., 5-15 such as 10-stacks). The host cells and the transfection mixture may be incubated in the presence of chloroquine. In a preferred embodiment, the host cells and the transfection mixture may be incubated in the absence of chloroquine for the benefits noted herein.

After transfection, the cells may be cultured for a suitable period, e.g., 4-24 hours, such as 4-6 hours or 12-18 hours. The culture medium can then be replaced with a second culture medium. In some instances, the second culture medium can be a complete medium, which may comprise DEME supplemented with FBS (e.g., about 2-5% such as 3%), 1% sodium pyruvate (e.g., about 0.5-3% such as 1%), benzonase (about 30-80 U/mL such as 50 U/ml), and one or more salts such as MgCl₂. In some embodiments, the methods disclosed herein do not use a conditioned medium in the medium change post transfection. Conditioned medium was found not required for high titer lentivirus manufacture. Its removal eliminates additional storage requirements, eliminates the need to mix fresh media and conditioned media, and improves harvest media quality as both the pH and amount of glucose present in the mixture of conditioned medium and fresh medium (typically complete culture media) was significantly lower than that in non-mixed complete culture media.

Alternatively, the second culture medium can be a mixture of conditioned medium and fresh medium containing about 8-12% FBS (e.g., 10%) at a suitable ratio, for example, 1:1, 2:1, 3:1, 4:1, 1:2, 1:3, 1:4. In one particular example, the ratio can be 1:1. Conditioned media refer to spent media harvested from cultured cells. They contain metabolites, growth factors, and extracellular matrix proteins secreted into the media by the cultured cells. The fresh medium can be any of the culture medium disclosed here, for example, a complete culture medium, which can be DEME (e.g., high glucose; 4,500 mg/L) in GlutaMAX-I and HEPES buffer supplemented with 10% Fetal Bovine Serum and 1 mM sodium pyruvate.

The transfected cells can be further cultured for a suitable period, e.g., 12 hours to 36 hours (e.g., around 15 to 18 hours). The culture supernatant can then be collected. Such supernatant contains lentiviral particles for further enrichment. In some embodiments, the supernatant can be collected about 28-45 hours (e.g., 32-42 hours) post transfection for only once (i.e., a single collection), as opposed to second collection/harvest adopted in conventional approaches. The elimination of a second collection/harvest of supernatant was found to improve lentivirus quality (e.g., improve the infectious: defective virus ratio) prior to further purification and concentration. It was found that the second harvest has less number of infectious particles compared to the first harvest, and contains more non-infectious particles. See Examples below. Hence higher quality vector is generated initially (harvest 1), and mixing harvest 1 and 2 may increase the defective particles in the lentivirus preparation. The timing of the single collection/harvest was optimized to get the best ratio of infectious:defective particles within the single collection/harvest.

The supernatant collection can then be passed through a leukocyte reduction filter, followed by a 0.45 μM filter. The passing through solution, which contains lentiviral particles, can be subject to further purification and concentration by, e.g., ion-exchange chromatography.

In some embodiments, a titer can be determined for the lentiviral particles described herein. In some embodiments, lentiviral particles herein can have a functional titer between about 1×10⁵ to 1×10²⁰ transduction units per ml (TU/ml) (e.g., about 1×10⁵, 1×10⁶, 1×10⁷, 1×10⁸, 1×10⁹, 1×10¹⁰, 1×10¹¹, 1×10¹², 1×10¹³, 1×10¹⁴, 1×10¹⁷, 1×10²⁰).
(c) Target Cells

Lentiviral transfer vectors and lentiviral particles of the present disclosure may be used to achieve inducible, constitutive, conditional, reversible, or tissue-specific expression in cells, tissues, or organisms. In certain embodiments, lentiviral transfer vectors and/or lentiviral particles disclosed herein can be used to achieve inducible, constitutive, conditional, reversible, or tissue-specific expression in at least one target cell. As used herein a “target cell,” refers to a cell that contains the nucleic acid sequence of interest that has been introduced into the cell by lentiviral transfer vectors and/or lentiviral particles disclosed herein. In certain embodiments herein, the term “target cell” refers to cultures of cells of various mammalian species. In some embodiments, the target cell is a mammalian cell. In some embodiments, the target cell might be a eukaryotic cell, a prokaryotic cell, an embryonic stem cell, a cancer cell, a neuronal cell, an epithelial cell, an immune cell, an endocrine cell, a muscle cell, an erythrocyte, or a lymphocyte. In some examples, a target cell may be a primary human cell. In some examples, a target cell may be an immortal human cell line. In some examples, a target cell may be a primary human cell obtained from a biological sample collected from a human. In some examples, a target cell may be a primary human cell obtained from a biopsy of a human solid tumor. In some embodiments, a target cell treated with lentiviral particles herein having a functional titer between about 1×10⁵ to 1×10²⁰ transduction units per ml (TU/ml) (e.g., about 1×10⁵, 1×10⁶, 1×10⁷, 1×10⁸, 1×10⁹, 1×10¹⁰, 1×10¹¹, 1×10¹², 1×10¹³, 1×10¹⁴, 1×10¹⁷, 1×10²⁰).

In some embodiments, target cells herein can be infected with lentiviral particles disclosed herein at about 0.1 to about 10 MOI (e.g., about 0.1, 0.2, 0.3, 0.4, 0.5, 1.0, 1.5, 2.0, 2.5, 3.0, 3.5, 4.0, 4.5, 5.0, 6.0, 7.0, 8.0, 9.0, 10 MOI). MOI (multiplicity of infection) is the number of viral particles that can infect each cell. In some embodiments, target cells herein can be infected with lentiviral particles disclosed herein so that each cell receives one allele.

In some embodiments, lentiviral transfer vectors and/or lentiviral particles disclosed herein can be used to achieve inducible expression of at least transgene of interest in a target cell. In some embodiments, expression of at least transgene of interest in a target cell can be induced by treating the target cell with tetracycline and/or doxycycline as described herein. In some embodiments, a transgene herein can have inducible expression of at least one known protein target of at least one biomolecule. In some embodiments, a transgene herein can have inducible expression of at least one known protein target of at least one small molecule. In some embodiments, a transgene herein can have inducible expression of at least one known protein target of at least one polypeptide (e.g., a protein, a peptide, an antibody). In some embodiments, a transgene herein can have inducible expression of at least one known protein target of at least one biologic. As used herein, a “biologic” can be a type of treatment that uses substances made from living organisms to treat disease. In some examples, a biologic can be a biomolecule as described herein. In some examples, a biologic can be a protein, a peptide, an antibody, or any combination thereof. In some examples, a biologic herein can be a cellular therapy product, a gene therapy product, or any combination thereof. Cellular therapy products can include, but are not limited to, cellular immunotherapies, cancer vaccines, and other types of both autologous and allogeneic cells for certain therapeutic indications, including hematopoietic stem cells and adult and embryonic stem cells. Human gene therapy can modify or manipulate the expression of a gene or to alter the biological properties of living cells for therapeutic use. Non-limiting examples of cellular and gene therapy products can include: ALLOCORD (HPC (Hematopoietic Progenitor Cell), Cord Blood); BREYANZI (lisocabtagene maraleucel); CLEVECORD (HPC Cord Blood); Ducord, HPC Cord Blood; GINTUIT (Allogeneic Cultured Keratinocytes and Fibroblasts in Bovine Collagen); HEMACORD (HPC, cord blood); IMLYGIC (talimogene laherparepvec); KYMRIAH (tisagenlecleucel); LAVIV (Azficel-T); LUXTURNA (Voretigene neparvovec); MACI (Autologous Cultured Chondrocytes on a Porcine Collagen Membrane); PROVENGE (sipuleucel-T); TECARTUS (brexucabtagene autoleucel); YESCARTA (axicabtagene ciloleucel); and ZOLGENSMA (onasemnogene abeparvovec-xioi). In some embodiments, a transgene herein can have inducible expression of at least one known protein target of at least one biomolecule used to treat a cancer. In some embodiments, a transgene herein can have inducible expression of at least one known protein target of at least one biologic used to treat a cancer.

Non-limiting examples of cancers that can be treated by at least biomolecule targeting a protein expressed by a transgene herein can include breast cancer, prostate cancer, lymphoma, skin cancer, pancreatic cancer, colon cancer, melanoma, malignant melanoma, ovarian cancer, brain cancer, primary brain carcinoma, head-neck cancer, glioma, glioblastoma, liver cancer, bladder cancer, non-small cell lung cancer, head or neck carcinoma, breast carcinoma, ovarian carcinoma, lung carcinoma, small-cell lung carcinoma, Wilms' tumor, cervical carcinoma, testicular carcinoma, bladder carcinoma, pancreatic carcinoma, stomach carcinoma, colon carcinoma, prostatic carcinoma, genitourinary carcinoma, thyroid carcinoma, esophageal carcinoma, myeloma, multiple myeloma, adrenal carcinoma, renal cell carcinoma, endometrial carcinoma, adrenal cortex carcinoma, malignant pancreatic insulinoma, malignant carcinoid carcinoma, choriocarcinoma, mycosis fungoides, malignant hypercalcemia, cervical hyperplasia, leukemia, acute lymphocytic leukemia, chronic lymphocytic leukemia, acute myelogenous leukemia, chronic myelogenous leukemia, chronic granulocytic leukemia, acute granulocytic leukemia, hairy cell leukemia, neuroblastoma, rhabdomyosarcoma, Kaposi's sarcoma, polycythemia vera, essential thrombocytosis, Hodgkin's disease, non-Hodgkin's lymphoma, soft-tissue sarcoma, mesothelioma, osteogenic sarcoma, primary macroglobulinemia, and retinoblastoma, and the like.

Non limiting examples of protein targets of biomolecules used to treat a cancer that can be expressed by the transgenes herein can include 2B4, 4-1BB, 4-1BBL, A33, adenosine A2a receptor, Akt, ALK, Androgen receptor, Ang-1, Ang-2, Annexin A3, Aurora A, Aurora B, B7-H3, B7-H4, Bcl-2, Bcr-Abl, BRAF, BTK, BTLA, BTN2A1, CA-125, CAIX, CCR4, CD105/endoglin, CD109, CD123, CD155, CD16, CD160, CD19, CD20, CD200, CD200R, CD22, CD24, CD25, CD27, CD28, CD30, CD33, CD36, CD37, CD38, CD40, CD40L, CD47, CD48, CD52, CD70, CD79b, CD80, CD86, CD96, CDK4, CDK6, CDK9, CEA, CEACAM1, ChK1, ChK2, c-KIT, c-Met/HGFR, COX2, CSF-1R, CSF2, CTLA-4, CXCR2, CXCR4, DDR2, DLL3, DLL4, DNAM-1, DR5, EGFR, EpCAM, EPHA3, EphB4, ERK1, ERK2/p38 MAPK, FAK, FAP, FGF-2, FGFR1, FGFR2, FGFR3, FGFR4, Flt-3, Gal-9, GITR, GITRL, Glypican-3, HDAC1, HDAC2, HDAC3, HDAC4, HDAC5, HDAC7, HDAC8, HDAC9, HER2, HER3, HER4/ERBB4, HGF, HHLA2, HIF-1α, HSP27, HSP90, HVEM, ICOS, ICOS Ligand, IDO, IGF1R, IL-13, IL-6, JAK1, JAK2, JAK3, KRAS, LAG-3, LIGHT, MDM2, MEK1, MEK2, MMP-1, MMP-10, MMP-11, MMP-13, MMP-2, MMP-7, MMP-9, mTOR, Mucin 1, Myc, NF-κB, NKG2A, NRAS, NTRK1, NTRK2, NTRK3, OX40, OX40L, p53, PAF, PARP1, PARP2, PD1, PDGFR-α, PDGFR-β, PD-L1, PD-L2, PI3Kα, PI3Kβ, PI3Kγ, PI3Kδ, PIM1, PIM3, PSMA, PTEN, RAF-1, RANKL, RET, S100A4, SIRPα, SLAMF7, SMO, Src, STAT3, STEAP-1, Syk, TDO, TGFβ, Tie-2, TIGIT, TIM-3, TLR8, TMIGD2, TNF-α, Toll-like receptor 3, TRAIL, TRAILR1, TROP-2, VEGF, VEGF-C, VEGFR-1, VEGFR-2, VEGFR-3, VISTA, γ-secretase, or any combination thereof.

### III. Methods

In certain embodiments, compositions disclosed herein can be used in methods of screening for biomolecule-resistant proteins. The term “biomolecule-resistant protein” as used herein can refer to a protein that fails to have the expected phenotypical response when targeted by a biomolecule. In some examples, a biomolecule-resistant protein can be a drug-resistant protein. In some embodiments, compositions disclosed herein can be used in methods of screening for drug-resistant protein targets. In some embodiments, compositions disclosed herein can be used in methods of screening for one or more protein mutations conferring resistance to a biomolecule.

In certain embodiments, methods herein can screen for one or more protein mutations conferring resistance to a biomolecule. In some embodiments, methods herein can screen for one or more nucleic acid mutations in a protein that are known to confer resistance to a biomolecule.

In some embodiments, methods herein can screen for one or more spontaneous mutations in a protein that confers resistance to a biomolecule. In some embodiments, one or more spontaneous mutations in a protein that confers resistance to a biomolecule can be created by a lentiviral reverse transcriptase encoded by any of the lentiviral transfer vectors and/or lentiviral particles disclosed herein. In some embodiments, a lentiviral reverse transcriptase encoded by any of the lentiviral transfer vectors and/or lentiviral particles disclosed herein can have one or more mutations to increase the rate of which the reverse transcriptase creates the spontaneous mutations that confer resistance to a biomolecule in a protein. In some embodiments, a lentiviral reverse transcriptase having a M230I mutation encoded by any of the lentiviral transfer vectors and/or lentiviral particles disclosed herein can increase the rate of which the reverse transcriptase creates the spontaneous mutations that confer resistance to a biomolecule in a protein. In some embodiments, a lentiviral reverse transcriptase having a M230I mutation and a Y501W mutation (D-RT), encoded by any of the lentiviral transfer vectors and/or lentiviral particles disclosed herein can increase the rate at which the reverse transcriptase creates the spontaneous mutations that confer resistance to a biomolecule in a protein.

In some embodiments, methods of screening for one or more spontaneous mutations in a protein that confers resistance to a biomolecule can encompass creation of the one or more spontaneous mutations during integration of a transgene into a target cell genome using any of the lentiviral transfer vectors and/or lentiviral particles disclosed herein. In some embodiments, methods of screening for one or more spontaneous mutations in a protein that confers resistance to a biomolecule can encompass creation of the one or more spontaneous mutations by a lentiviral reverse transcriptase during integration of a transgene into a target cell genome using the any of the lentiviral transfer vectors and/or lentiviral particles disclosed herein. In some embodiments, methods of screening for one or more spontaneous mutations in a protein that confers resistance to a biomolecule can encompass creation of the one or more spontaneous mutations by a lentiviral reverse transcriptase having at least one mutation during integration of a transgene into a target cell genome using the any of the lentiviral transfer vectors and/or lentiviral particles disclosed herein. In some embodiments, methods of screening for one or more spontaneous mutations in a protein that confers resistance to a biomolecule can encompass creation of the one or more spontaneous mutations by a lentiviral reverse transcriptase having a M230I mutation during integration of a transgene into a target cell genome using the any of the lentiviral transfer vectors and/or lentiviral particles disclosed herein. In some embodiments, methods of screening for one or more spontaneous mutations in a protein that confers resistance to a biomolecule can encompass creation of the one or more spontaneous mutations by a lentiviral reverse transcriptase having a M230I mutation and a Y501W mutation during integration of a transgene into a target cell genome using the any of the lentiviral transfer vectors and/or lentiviral particles disclosed herein.

In some embodiments, methods herein of creating one or more spontaneous mutations in a protein that confers resistance to a biomolecule during integration of a transgene into a target cell genome can use any of the lentiviral transfer vectors and/or lentiviral particles disclosed herein to introduce at least one mutation in the protein's amino acid sequence. In some embodiments, methods herein of creating one or more spontaneous mutations in a protein that confers resistance to a biomolecule during integration of a transgene into a target cell genome can use any of the lentiviral transfer vectors and/or lentiviral particles disclosed herein to introduce at least one mutation in the protein's nucleic acid sequence.

In some embodiments, a biomolecule-resistant protein subject to methods herein can have at least one mutation resulting from single base substitutions in its nucleic acid sequence. In some examples, a biomolecule-resistant protein subject to methods herein can have one or more of the following six substitution subtypes: C>A, C>G, C>T, T>A, T>C, and T>G (all substitutions are referred to by the pyrimidine of the mutated Watson-Crick base pair). In some embodiments, substitution subtypes C>A can have a sequence context of ACA, ACC, ACG, ACT, CCA, CCC, CCG, CCT, GCA, GCC, GCG, GCT, TCA, TCC, TCG, or TCT. In some embodiments, substitution subtypes C>G can have a sequence context of ACA, ACC, ACG, ACT, CCA, CCC, CCG, CCT, GCA, GCC, GCG, GCT, TCA, TCC, TCG, or TCT. In some embodiments, substitution subtypes C>T can have a sequence context of ACA, ACC, ACG, ACT, CCA, CCC, CCG, CCT, GCA, GCC, GCG, GCT, TCA, TCC, TCG, or TCT. In some embodiments, substitution subtypes T>A can have a sequence context of ATA, ATC, ATG, ATT, CTA, CTC, CTG, CTT, GTA, GTC, GTG, GTT, TTA, TTC, TTG, or TTT. In some embodiments, substitution subtypes T>C can have a sequence context of ATA, ATC, ATG, ATT, CTA, CTC, CTG, CTT, GTA, GTC, GTG, GTT, TTA, TTC, TTG, or TTT. In some embodiments, substitution subtypes T>G can have a sequence context of ATA, ATC, ATG, ATT, CTA, CTC, CTG, CTT, GTA, GTC, GTG, GTT, TTA, TTC, TTG, or TTT.

In some embodiments, methods of screening for one or more spontaneous mutations in a protein that confers resistance to a biomolecule can encompass creation of the one or more spontaneous mutations during integration of a transgene into a target cell genome using the any of the lentiviral transfer vectors and/or lentiviral particles disclosed herein and further inducing expression of the protein from the transgene. In some embodiments, methods of screening for one or more spontaneous mutations in a protein that confers resistance to a biomolecule can encompass creation of the one or more spontaneous mutations during integration of a transgene into a target cell genome using the any of the lentiviral transfer vectors and/or lentiviral particles disclosed herein and further inducing expression of the protein from the transgene by activating the inducible promoter included in any of the lentiviral transfer vectors described herein. In some embodiments, methods of screening for one or more spontaneous mutations in a protein that confers resistance to a biomolecule can encompass creation of the one or more spontaneous mutations during integration of a transgene into a target cell genome using the any of the lentiviral transfer vectors and/or lentiviral particles disclosed herein and further inducing expression of the protein from the transgene by treating the transduced target cells with tetracycline and/or doxycycline as described herein.

In some embodiments, methods of screening for one or more spontaneous mutations in a protein that confers resistance to a biomolecule can encompass creation of the one or more spontaneous mutations during integration of a transgene into a target cell genome using the any of the lentiviral transfer vectors and/or lentiviral particles disclosed herein and further treating the transduced target cell with the biomolecule.

In some embodiments, screening methods herein include further treating the transduced target cell with a biomolecule at a concentration known to confer a selectable phenotype. In some embodiments, a selectable phenotype can be cell viability, one or more cellular functions (e.g., enzymatic function, protein production), cellular proliferation, and the like. In some embodiments, a selectable phenotype can result from activation, repression, or any combination thereof of one or more reporter genes encompassed in the lentiviral transfer vectors and/or lentiviral particles disclosed herein.

In some embodiments, screening methods herein include further treating the transduced target cell with a biomolecule at a concentration known to kill between about 50% to about 100% (e.g., about 50%, 60%, 70%, 80%, 85%, 90%, 95%, 99%, 100%) of viable target cells. In some embodiments, screening methods herein include further treating the transduced target cell with a biomolecule at a concentration known to repress one or more reporter genes of the lentiviral transfer vectors and/or lentiviral particles disclosed herein. In some embodiments, screening methods herein include further treating the transduced target cell with a biomolecule at a concentration known to repress one or more reporter genes by about 50% to about 100% (e.g., about 50%, 60%, 70%, 80%, 85%, 90%, 95%, 99%, 100%). In some embodiments, screening methods herein include further treating the transduced target cell with a biomolecule at a concentration known to repress one or more fluorescent reporter genes of the lentiviral transfer vectors and/or lentiviral particles disclosed herein. In some embodiments, screening methods herein include further treating the transduced target cell with a biomolecule at a concentration known to decrease fluorescence by about 50% to about 100% (e.g., about 50%, 60%, 70%, 80%, 85%, 90%, 95%, 99%, 100%).

In some embodiments, a biomolecule for use in the screening methods herein can be a treatment for a disease. In some embodiments, a biomolecules used for a treatment of a disease or a condition can be a gene therapy, an immunotherapy, a biologic, an antibody, a small molecule, a drug, and the like. In some embodiments, a biomolecule for use in the screening methods herein can be known to target a protein encoded by the transgene of the present disclosure, wherein the protein can be known to be involved in a disease or condition. Non-limiting examples of diseases having the biomolecules and/or proteins for use in the methods described herein can be an autoimmune disorder (e.g., rheumatoid arthritis, lupus erythematosus, type 1 diabetes), a metabolic disorder (e.g., type 2 diabetes), a bacterial infection, a viral infection, a cancer, or the like. In some embodiments, a disease having the biomolecules and/or proteins for use in the methods described herein can be cancer.

In some embodiments, methods of screening for one or more spontaneous mutations in a protein that confers resistance to a biomolecule can encompass creation of the one or more spontaneous mutations during integration of a transgene into a target cell genome using the any of the lentiviral transfer vectors and/or lentiviral particles disclosed herein, treating the transduced target cell with the biomolecule, and further harvesting nucleic acids from transduced target cells that confer a selectable phenotype after treatment with the biomolecule. In some embodiments, methods of screening for one or more spontaneous mutations in a protein that confers resistance to a biomolecule can encompass creation of the one or more spontaneous mutations during integration of a transgene into a target cell genome using the any of the lentiviral transfer vectors and/or lentiviral particles disclosed herein, treating the transduced target cell with the biomolecule, and further harvesting nucleic acids from transduced target cells that remain viable after treatment with the biomolecule. In some embodiments, nucleic acids harvested from transduced target cells that remain viable after treatment with the biomolecule can be subjected to genomic sequencing. Non-limiting examples of sequencing chemistries that can be used herein can include Sanger sequencing, pyrosequencing, reversible terminator chemistry, sequencing by ligation, H+ Ion sensitive transistor, nanopore sequencing, and the like. In some embodiments, nucleic acids harvested from transduced target cells that remain viable after treatment with the biomolecule can be subjected to next generation sequencing (NGS). In some embodiments, genomic sequencing results from the nucleic acids harvested from transduced target cells that remained viable after treatment with the biomolecule can be compared with a known, unmutated nucleic acid sequence for the protein of interest to determine if one or more mutations are present in the proteins of the transduced target cells.

### IV. Kits

In certain embodiments, the present disclosure provides for kits having one or more of the lentiviral vectors disclosed herein. In some embodiments, the present disclosure provides for kits having the compositions disclosed herein to perform any one of the methods disclosed herein (e.g., screening methods). In some embodiments, kits herein can have one or more lentiviral vectors and/or one or more lentiviral particles for use in the methods disclosed herein. In some embodiments, a kit may have any of a number of additional components or reagents in any combination. The various combinations are not set forth explicitly, but each combination is included in the scope of the invention. For example, one or more of the following items: (i) one or more vectors, e.g., plasmids, that collectively comprise nucleic acid sequences coding for retroviral or lentiviral Gag and Pol proteins and an envelope protein. The set of vectors may include two or more vectors. According to certain embodiments of the invention the kit includes (in addition to a lentiviral vector of the invention) at least two vectors (e.g., plasmids), one of which provides nucleic acid sequences coding for Gag and Pol and the other of which provides nucleic acid segments coding for an envelope protein; (ii) cells (e.g., a cell line) that are permissive for production of lentiviral particles (e.g., 293T cells); (iii) packaging cells, e.g., a cell line that is permissive for production of lentiviral particles and provides the proteins Gag, Pol, Env; (iv) cells suitable for use in titering lentiviral particles; (v) a lentiviral vector comprising a heterologous nucleic acid segment such as a reporter gene that may serve as a positive control (referred to as a “positive control vector”); (vi) an agent/compound suitable for inducing transgene expression (e.g., tetracycline, doxycycline).

In certain embodiments, kits can include instructions for use of lentiviral vectors in the methods disclosed herein. Instructions may, for example, include protocols and/or describe conditions for transfection, transduction, infection, production of lentiviral particles, gene silencing, etc. In some embodiments, kits can include one or more vessels or containers so that some or all of the individual components and reagents may be separately housed. Kits may also include a means for enclosing individual containers in relatively close confinement for commercial sale, e.g., a plastic box, in which instructions, packaging materials such as styrofoam, etc., may be enclosed. An identifier, e.g., a bar code, radio frequency identification (ID) tag, etc., may be present in or on the kit or in or one or more of the vessels or containers included in the kit. An identifier can be used, e.g., to uniquely identify the kit for purposes of quality control, inventory control, tracking, movement between workstations, etc.

### General Techniques

The practice of the present disclosure will employ, unless otherwise indicated, conventional techniques of molecular biology (including recombinant techniques), microbiology, cell biology, biochemistry, and immunology, which are within the skill of the art. Such techniques are explained fully in the literature, such as Molecular Cloning: A Laboratory Manual, second edition (Sambrook, et al., 1989) Cold Spring Harbor Press; Oligonucleotide Synthesis (M. J. Gait, ed. 1984); Methods in Molecular Biology, Humana Press; Cell Biology: A Laboratory Notebook (J. E. Cellis, ed., 1989) Academic Press; Animal Cell Culture (R. I. Freshney, ed. 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds. 1993-8) J. Wiley and Sons; Methods in Enzymology (Academic Press, Inc.); Handbook of Experimental Immunology (D. M. Weir and C. C. Blackwell, eds.): Gene Transfer Vectors for Mammalian Cells (J. M. Miller and M. P. Calos, eds., 1987); Current Protocols in Molecular Biology (F. M. Ausubel, et al. eds. 1987); PCR: The Polymerase Chain Reaction, (Mullis, et al., eds. 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: a practice approach (D. Catty., ed., IRL Press, 1988-1989); Monoclonal antibodies: a practical approach (P. Shepherd and C. Dean, eds., Oxford University Press, 2000); Using antibodies: a laboratory manual (E. Harlow and D. Lane (Cold Spring Harbor Laboratory Press, 1999); The Antibodies (M. Zanetti and J. D. Capra, eds. Harwood Academic Publishers, 1995); DNA Cloning: A practical Approach, Volumes I and II (D. N. Glover ed. 1985); Nucleic Acid Hybridization (B. D. Hames & S. J. Higgins eds. (1985»; Transcription and Translation (B. D. Hames & S. J. Higgins, eds. (1984»; Animal Cell Culture (R. I. Freshney, ed. (1986»; Immobilized Cells and Enzymes (IRL Press, (1986»; and B. Perbal, A practical Guide To Molecular Cloning (1984); F. M. Ausubel et al. (eds.).

Having described several embodiments, it will be recognized by those skilled in the art that various modifications, alternative constructions, and equivalents may be used without departing from the spirit of the present inventive concept. Additionally, a number of well-known processes and elements have not been described in order to avoid unnecessarily obscuring the present inventive concept. Accordingly, this description should not be taken as limiting the scope of the present inventive concept.

Those skilled in the art will appreciate that the presently disclosed embodiments teach by way of example and not by limitation. Therefore, the matter contained in this description or shown in the accompanying drawings should be interpreted as illustrative and not in a limiting sense. The following claims are intended to cover all generic and specific features described herein, as well as all statements of the scope of the method and assemblies, which, as a matter of language, might be said to fall there between.

### EXAMPLES

The following examples are included to demonstrate preferred embodiments of the disclosure. It should be appreciated by those of skill in the art that the techniques disclosed in the examples that follow represent techniques discovered by the inventor to function well in the practice of the present disclosure, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the present disclosure.

### Example 1

Lentivirus production. All cDNAs were cloned into the doxycycline-inducible lentiviral expression plasmid pLVX-TRE3G-IRES. CRISPR-Cas9 viruses were made from pLentiCRISPRv2 with the following gRNA sequences:

gBCR-ABL-NTCD-1:
(SEQ ID NO: 3)
CACCGCTACTCCCCCCGGAGTTTTG,
gBCR-ABL-NTCD-2:
(SEQ ID NO: 4)
CACCGAGGGAGAGCGGCTTTTGTCC,
gBCR-ABL-NTCD-3:
(SEQ ID NO: 5)
CACCGAGAGGAGAAGTCCTCCTCGC,
gBCR-ABL-sv11:
(SEQ ID NO: 6)
CACCGAAGGAGCTGCTGCGTTTGGG

To generate lentiviral particles, 1.5 million Lenti-X 293T cells (Clontech, catalog #632180) were forwarded transfected with 9 μg pCMV-dR8.91 (lentiviral packaging plasmid), 3 μg pMD2.G (lentiviral envelope plasmid) and 3 μg of pLVX-TRE3G-IRES, pLVX-EF1a-Tet3G, or pLentiCRISPRv2 using FuGene6 (Promega, catalog #E2691) following the manufacturers protocol. After 12 hours, media was changed and viral supernatant was collected every day for 3 days and filtered through a 45 micron syringe filter (Corning, catalog #431220). When necessary, multiple lentiviral preps were pooled together and concentrated using Lenti-X Concentrator (Takara, catalog #631231). Cells were then transduced using the lentiviral supernatant and 6 μg/mL polybrene (Santa Cruz, catalog #sc-134220)

Expression vectors. All cDNAs were cloned into the doxycycline-inducible lentiviral expression plasmid pLVX-TRE3G-IRES. RUVBL1 cDNA was reverse transcribed (SuperScript III, ThermoFisher catalog #18080051) from the H2009 cell line. EGFRdel746-750 cDNA was reverse transcribed from the HCC827 cell line. The HIV RT-M230I mutant (M-RT) was generated by site-directed mutagenesis of codon 230 (ATG→ATC) in the reverse transcriptase in the plasmid pCMV-dR8.91 with the Q5 Site-Directed Mutagenesis Kit (New England BioLabs, catalog #E0554S). The double mutant M230I and Y501W (D-RT) was similarly generated. The BCR-ABL open reading frame was PCR amplified from the plasmid MSCV-(pBabemcs)-humanp210BCR-ABL-IRES-GFP (Addgene plasmid #79248; RRID:Addgene_79248). For NTCD, the nucleotides 907-1122 were removed. KRAS-WT and KRAS-G12C cDNA were chemically synthesized using a codon-optimized reading frame (sequence shown below) because use of the endogenous cDNA sequence from H358 resulted in extremely low expression.

KRAS-WT (SEQ ID NO: 7):
ATGACAGAATATAAACTGGTAGTAGTAGGTGCAGGTGGAG
TTGGAAAATCCGCTCTTACAATTCAACTCATCCAAAACCA
CTTCGTCGATGAGTACGACCCCACTATCGAAGACAGTTAT
AGAAAACAGGTCGTTATCGACGGCGAGACGTGCCTTCTCG
ACATCCTGGATACCGCCGGCCAGGAAGAATATAGCGCCAT
GCGGGATCAATATATGCGTACAGGCGAAGGGTTCCTGTGC
GTGTTCGCAATTAACAACACGAAGTCCTTCGAGGACATCC
ATCACTACCGGGAGCAGATCAAGCGTGTGAAAGATAGCGA
GGACGTCCCGATGGTGCTTGTGGGCAACAAGTGCGATCTC
CCAAGCCGAACTGTGGATACCAAGCAAGCACAAGATCTGG
CTCGGTCCTACGGCATCCCCTTCATCGAGACGTCTGCCAA
AACGAGGCAAGGCGTAGACGACGCATTTTACACGCTGGTG
AGAGAGATCCGCAAGCACAAGGAGAAAATGTCTAAGGACG
GAAAGAAGAAGAAGAAGAAGAGCAAAACGAAATGCGTGAT
CATGTAG
KRAS-G12C (SEQ ID NO: 8):
ATGACAGAATATAAATTGGTCGTTGTTGGAGCTTGTGGTG
TCGGGAAATCTGCACTTACCATTCAATTGATACAAAACCA
CTTCGTAGATGAGTACGACCCTACCATTGAAGACTCTTAT
AGAAAACAGGTTGTGATAGACGGGGAGACATGCCTGCTTG
ACATCCTGGATACTGCGGGACAGGAAGAATATTCAGCCAT
GCGCGATCAATATATGCGGACAGGCGAAGGGTTCCTGTGC
GTGTTCGCTATTAACAACACAAAGAGCTTCGAGGACATAC
ATCACTACCGTGAGCAGATCAAGCGGGTGAAAGATTCAGA
GGACGTGCCCATGGTTCTCGTGGGCAACAAGTGCGACCTT
CCCAGTAGGACTGTCGATACGAAGCAAGCCCAAGATCTGG
CCCGGAGCTACGGTATCCCATTCATCGAGACTAGCGCTAA
AACTCGGCAAGGCGTGGACGACGCATTTTACACTCTCGTC
CGCGAGATCAGAAAGCACAAGGAGAAAATGTCTAAGGACG
GCAAGAAGAAGAAGAAGAAATCCAAAACCAAATGCGTGAT
CATGTGA

Performing LentiMutate to identify drug-resistance mutations. To perform LentiMutate millions of cells are transduced with approximately one lentiviral integration per cell (e.g., transduction performed at a multiplicity of infection of approximately 0.3), with greater numbers of cells being transduced for larger DNA/cDNA cargos to saturate the possible mutagenic space. It was estimated that the M-RT makes roughly 1 error per 1500 nucleotides; thus, on average, to ensure that each base can be mutated to all other bases, for a 1500 base pair (bp) cargo, at least 4500 cells (1500×3=4500) should be infected. However due to biases, this number was multiplied by 100 (4500×100=450,000 cells). Then, to ensure that the rarest mutations are made multiple times, this number was multiplied by 5 (450,000×5=2,250,000). Thus, to perform LentiMutate on a 1500 bp cargo, transducing at least 2,250,000 cells was recommended and this number of cells must be maintained throughout the entire experiment. For a 3000 bp cargo, at least 4,500,000 cells were needed to be infected, and so on. Greater numbers of infected cells yielded more reliable and reproducible results.

For these examples herein, cells containing TET3G (pLVX-EF1a-Tet3G) were transduced at an MOI of 0.3, such that for pLVX-TRE3G-EGFRdel746-750-IRES 2 million cells were infected, for pLVX-TRE3G-RUVBL1-IRES 500,000 cells were infected, for pLVX-TRE3G-BCR-ABL-IRES 15 million cells were infected and for pLVX-TRE3G-KRAS-(G12C and WT) 3 million cells were infected. After infection and puromycin selection, for compound B experiments 500,000 H1993 cells/dish were plated into 4 15 cm dishes. Two plates received 1 μg/mL doxycycline for two days, and after two days 500 nM compound B+/− doxycycline (to respective plates) was added. Media was changed every three days until large resistant colonies emerged in the plates with doxycycline. For gefitinib/osimertinib/AMG 510 experiments, after infection and puromycin selection 2 million cells/dish were plated into 4 15 cm dishes. Two plates received 1 μg/mL doxycycline for two days, and after two days 1 μM gefitinib, 100 nM osimertinib or 1 μM AMG 510 and +/− doxycycline (to respective plates) was added. Media was changed every three days until resistant colonies emerged in the plates with doxycycline. These cells were then trypsinized and split 1:2 with continued drug treatment to aid in the outgrowth of true resistant cells from persistors. For imatinib experiments, after infection and selection 15 million cells/dish were plated into 2 T175s. One flask received 1 μg/mL doxycycline for two days, and after two days 1 μM imatinib+/− doxycycline was added to respective plates. Cells treated with doxycycline+imatinib were pelleted after ˜21 days of treatment. For all experiments, to identify resistance mutations cells that survived drug treatment in the drug+ doxycycline groups and the parental cell line were harvested, and genomic DNA was isolated (Qiagen DNeasy Blood & Tissue Kit, catalog #69504). For EGFR the entire open reading frame, for BCR-ABL two large amplicons, for RUVBL1 three short barcoded amplicons, and for KRASwt/KRASG12C two short barcoded amplicons were then PCR amplified, purified (Qiagen QIAquick PCR purification kit catalog #28104), ran on a D5000 HS ScreenTape on the Agilent Tapestation 4200 and DNA concentrations determined by Qubit. For EGFR and BCR-ABL the PCR products were sheared on the Covaris S2 for 40 seconds and libraries are prepared with the KAPA Hyper Prep Kit. Samples were end repaired, 3′ ends were adenylated and then barcoded with multiplex adapters. Samples are size selected prior to PCR to remove small fragments. The short RUVBL1 and KRASwt/KRASG12C PCR products were directly (i.e., without sonication) ligated with the adapters. Four cycles of PCR were used to amplify the libraries, which were then purified with AmpureXP beads and validated on the Agilent Tapestation 4200. Before being normalized and pooled, samples were quantified by Qubit then ran using the Nextseq500 V2.5 150PE or MiSeq V2.5 250/300PE chemistry. Reads obtained from pooled samples were separated into sample specific readlDs based on the barcode information. For each read in the pooled sample fastq file, the barcode and primer sequences were trimmed from the 5′ end. Using the trimmed fastq file and readlDs obtained in the first step, sample specific fastq files were generated using BBMap (v 38.11). These reads were mapped to the reference cDNA sequence using BWA (v. 0.7.15). For identification of point mutations, read pileup information was obtained from mapped reads using the samtools (v.0.1.19) mpileup command. From the read pileup information, per base mismatch/indel information was obtained using pileup2base tool. Per base error ratios were computed using an in-house Perl script, and the frequency of mutations at positions was compared between the parental (no doxycycline, no drug) and + doxycycline/drug surviving cells. For identification of deletions, the trimmed reads were mapped to the reference cDNA sequences using speedseq, which in turn used BWA-mem algorithm to map the reads. In addition to the alignment, speedseq tool marked duplicates and identified split and discordant read pairs. The speedseq mapped files were used to call structural variations (SVs) using Lumpy SV caller. Lumpy SV calls were further filtered using number of split and discordant supporting reads.

### Example 2

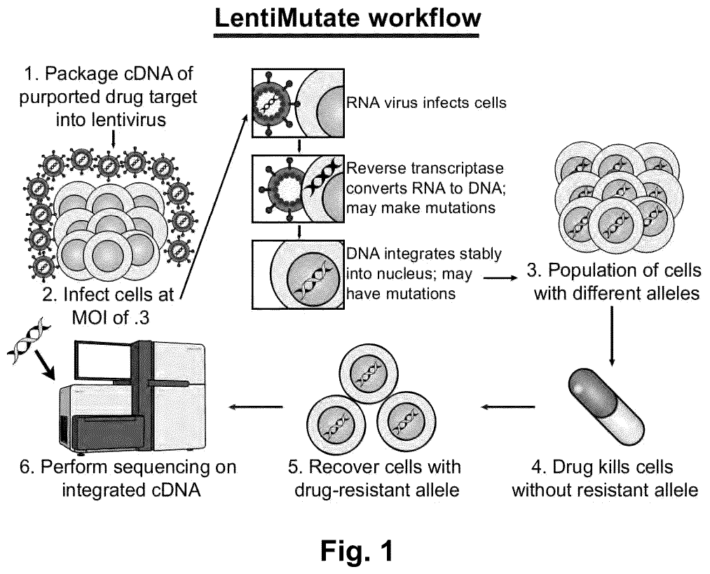

RUVBL1 and RUVBL2 are highly conserved ATPases that belong to the AAA+(ATPases Associated with various cellular Activities) superfamily and are involved in various complexes and cellular processes, several of which are closely linked to oncogenesis. A RUVBL1/2 inhibitor has been developed as a possible treatment for cancer. To identify the presence of any RUVBL1/2-inhibitor resistance mutations in the targeted RUVBL1/2, the following methods were used as generally depicted in FIG. 1.

First, RUVBL1 cDNA was cloned into a doxycycline-inducible lentiviral expression vector. Next, lentiviral particles were produced by co-transfecting HEK293T cells with the doxycycline-inducible lentiviral expression vector for RUVBL1 and the packaging vectors pCMV-dR8.91 and pMD2-VSV-G. Vectors were incubated with the HEK293T cells for about 72 hours in a 37° C. humidified incubator containing 5% CO₂. The supernatants were then harvested and filtered through 0.45-μm pore size filters. The viral vector titer was determined by transduction of non-small cell lung cancer target cells with serially diluted culture supernatants, treating with puromycin for 3 days, and counting the number of surviving cells.

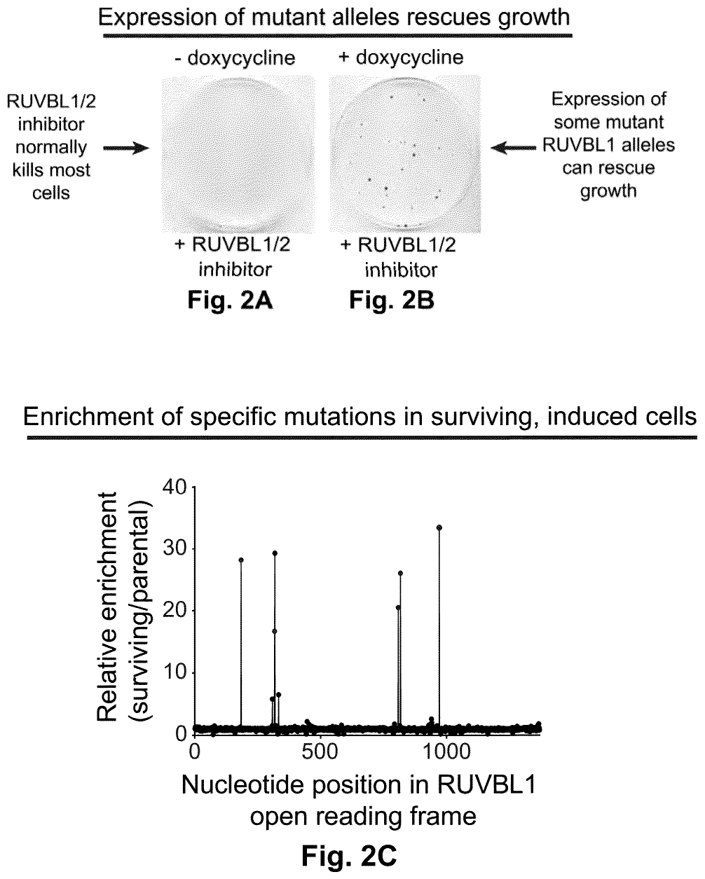

Once the titer was determined, lentiviral particles were used to infect 500,000 human non-small cell lung cancer (NSCLC) cells, such that each cell had only 1 integration (i.e., one exogenous allele of RUVBL1). The Multiplicity Of Infection (MOI)—the ratio of the number of transducing lentiviral particles to the number of cells—to accomplish this is about 0.3 MOI. Once transduced, NSCLC cells were treated with vehicle (control) or doxycycline to induce expression of the RUVBL1 alleles. Dishes that were full of NSCLC cells (confluent) were treated with a concentration of RUVBL1/2 inhibitor that was known to kill >99% of uninduced NSCLC cells (i.e., NSCLC cells that were not expressing these exogenous alleles). As shown in FIG. 2A, addition of the RUVBL1/2 inhibitor killed most of the NSCLC cells transduced with the lentiviral particles that were treated with vehicle (−doxycycline). In FIG. 2B however, NSCLC cells transduced with the lentiviral particles and treated with doxycycline had the outgrowth of multiple drug-resistant colonies. These data suggested that expression of some mutant RUVBL1 alleles can rescue cell growth from RUVBL1/2 inhibition.

To identity what mutations are enriched by RUVBL1/2-inhibitor treatment to promote cell survival, DNA was harvested from the cells shown in FIG. 2B and subjected to next-generation sequencing. As shown in FIG. 2C, the RUVBL1 expressed in the surviving NSCLC cells were enriched in the open reading frame mutations shown in Table 2 having a normalized mutation rates over at least 6:

TABLE 2

| Nucleotide | | Nucleotide | | Fold- | |
|---|---|---|---|---|---|
| position | Codon | mutation | Amino Acid Change | enriched | |
| 184 | 62 | G → A | alanine → threonine | 28 | fold |
| 317 | 106 | T → A | isoleucine → asparagine | 17 | fold |
| 328 | 106 | T → G | isoleucine → methionine | 29 | fold |
| 333 | 111 | G → A | no change | 6 | fold |
| 808 | 270 | G → A | glutamic acid → lysine | 21 | fold |
| 817 | 273 | G → A | aspartic acid → asparagine | 26 | fold |
| 917 | 324 | C → T | alanine → valine | 33 | fold |

Epidermal growth factor receptor (EGFR) inhibitors are used clinically for treatment of non-small cell lung cancer in some patients. Gefitinib is an EGFR inhibitor which interrupts signaling through the epidermal growth factor receptor (EGFR) in target cells. As such, Gefitinib is only effective in cancers with mutated and overactive EGFR. Unfortunately, some patients treated with Gefitinib develop T790M mutations in EGFR, which confer resistance to Gefitinib.

Following the surprising findings detailed above, a similar method was repeated. Briefly, EGFR cDNA was cloned into a doxycycline-inducible lentiviral expression vector and then co-transfecting HEK293T cells with the packaging vectors pCMV-dR8.91 and pMD2-VSV-G to produce lentiviral particles. Lentiviral particles were collected, and the titer was determined. Lentiviral particles were used to infect 500,000 human NSCLC cells, such that each cell had only 1 integration (i.e., one exogenous allele of EGFR). Once transduced, NSCLC cells were treated with vehicle (control) or doxycycline to induce expression of the EGFR alleles. Dishes that were full of NSCLC cells (confluent) were treated with a concentration of Gefitinib that was known to kill >99% of uninduced NSCLC cells.

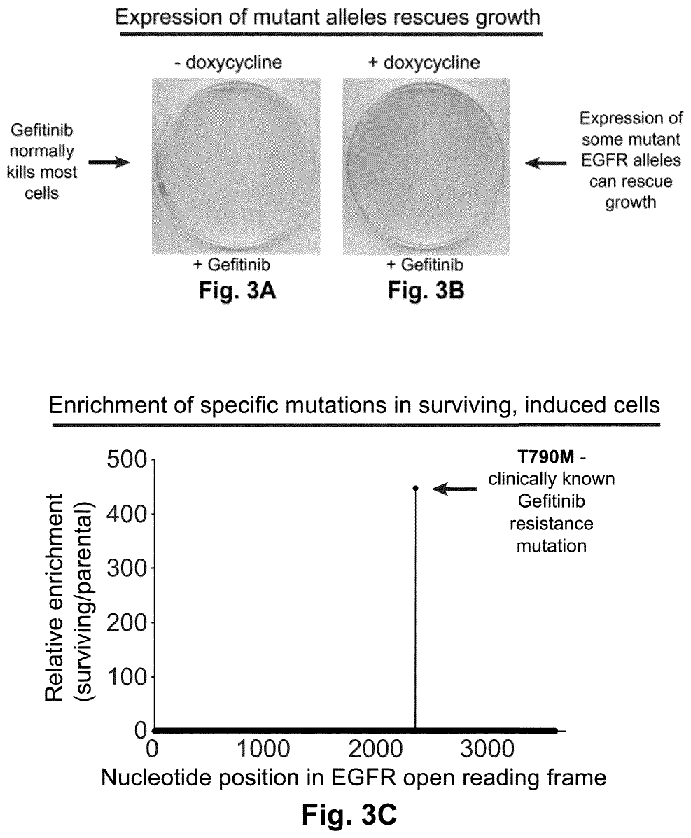

As shown in FIG. 3A, addition of Gefitinib killed most of the NSCLC cells transduced with the lentiviral particles that were treated with vehicle (−doxycycline). In FIG. 3B however, NSCLC cells transduced with the lentiviral particles and treated with doxycycline had some surviving cells following the addition of Gefitinib. DNA was harvested from the cells shown in FIG. 3B and subjected to next-generation sequencing. As shown in FIG. 3C, EGFR expressed in the surviving NSCLC cells following Gefitinib treatment were enriched for the T790M.

Accordingly, the methods provided in at least in this example herein of creating mutations during lentiviral transduction and identifying mutations that confer an interesting, selectable property are clinically relevant and can be used to predict drug resistance. These novel methods are referred to as “LentiMutate” in the present disclosure.

### Example 3

Lentiviruses express reverse transcriptase, which converts the viral RNA to double stranded DNA, and integrase, which inserts this viral DNA into the host DNA. A mutation, M230I, increases the error rate in the lentiviral reverse transcriptase. If the lentiviral reverse transcriptase used in LentiMutate was more error-prone, had increased mutational bias, or both the number of phenotypically relevant mutations could be maximally captured. Accordingly, a recombinant form of a lentiviral reverse transcriptase having the M230I was generated according to standard methods of molecular biology. Next, the LentiMutate method was performed similar to that described in Example 1 except that either a M230I mutated lentiviral reverse transcriptase (“M-RT”) or an unmutated, wild-type lentiviral reverse transcriptase (“WT-RT”) was used.

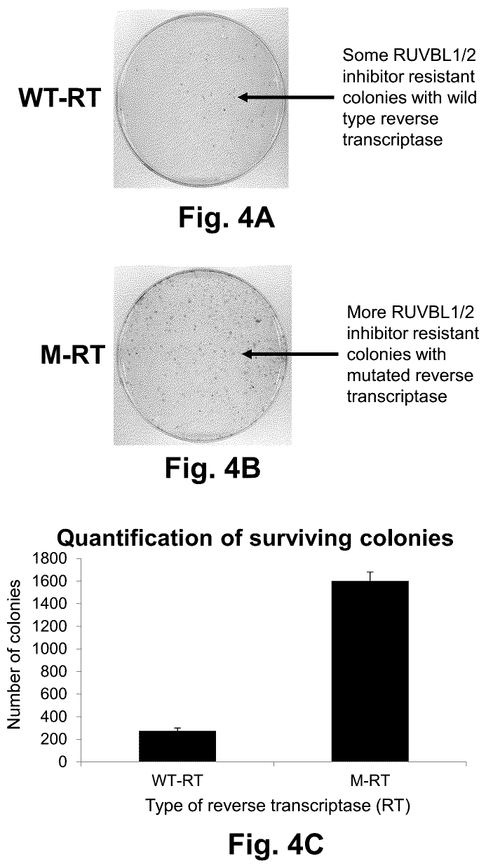

First, the lentivirus for doxycycline-inducible expression of RUVBL1 was packaged as described above using either WT-RT or M-RT and used to infect HEK293T cells. The resulting lentiviral particles were harvested from HEK293T cells and used to infect NSCLC cells. NSCLC cells were treated with doxycycline to induce expression of the RUVBL1 alleles. Confluent NSCLC cells were then treated with a concentration of RUVBL1/2 inhibitor that was known to kill >99% of uninduced NSCLC cells. As shown in FIG. 4A, some RUVBL1/2-inhibitor resistant cells (“colonies”) were present when infected with lentivirus having a wild type reverse transcriptase. FIG. 4B shows that more RUVBL1/2-inhibitor resistant colonies were present when infected with lentivirus having the mutated reverse transcriptase. Colonies from each plate were quantified and the use of M-RT in the LentiMutate method increased the number of RUVBL1/2-inhibitor resistant colonies by about 85% (FIG. 4C).

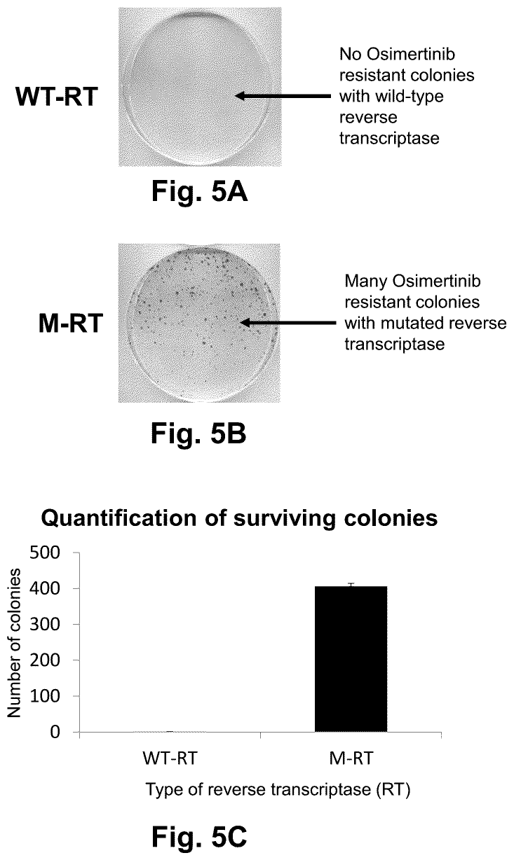

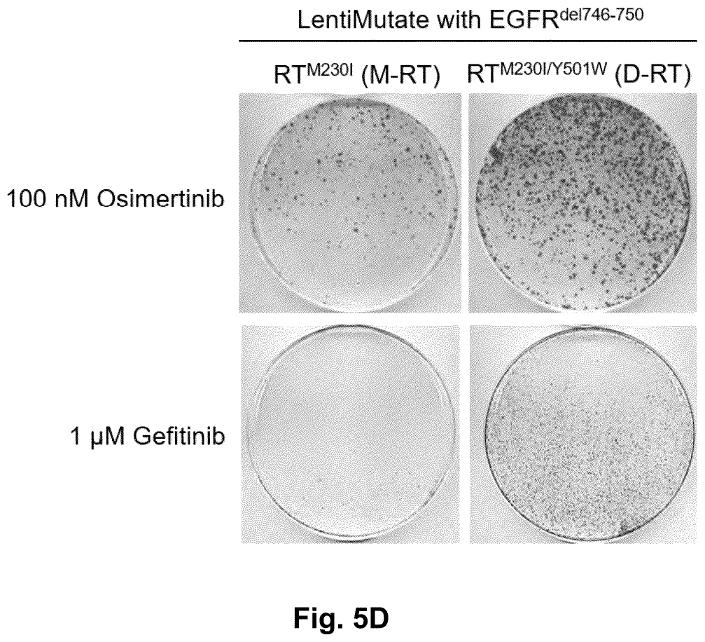

As an additional test, the lentivirus for doxycycline-inducible expression of EGFR was packaged as described above using either WT-RT or M-RT and used to infect HEK293T cells. The resulting lentiviral particles were harvested from HEK293T cells and used to infect NSCLC cells. NSCLC cells were treated with doxycycline to induce expression of the EGFR alleles. Confluent NSCLC cells were then treated with a concentration of Osimertinib, an EGFR inhibitor, that was known to kill >99% of uninduced NSCLC cells. As shown in FIG. 5A, no Osimertinib-resistant cells (“colonies”) were present when infected with lentivirus having a wild type reverse transcriptase. FIG. 5B shows several Osimertinib-resistant colonies were present when infected with a lentivirus having the mutated reverse transcriptase. Colonies from each plate were quantified and the use of M-RT in the LentiMutate method increased the number of RUVBL1/2-inhibitor resistant colonies by about 400% (FIG. 5C).

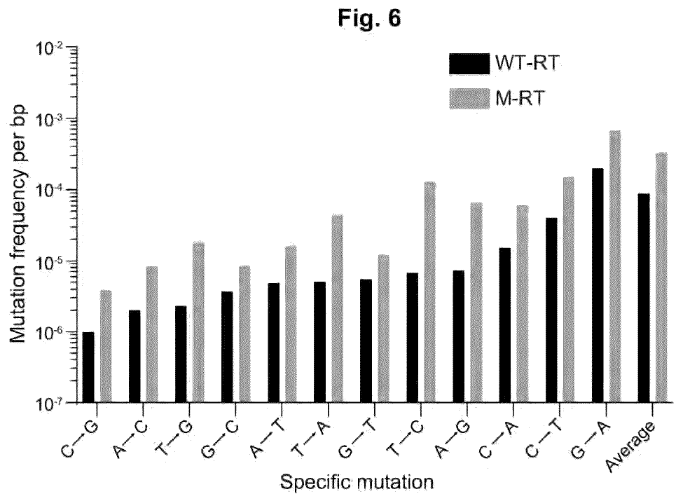

Next, the cargo DNA delivered into the target NSCLC cells by lentivirus having either WT-RT or M-RT was subjected to single molecule sequencing. During lentiviral transduction, which is a way to deliver “cargo” DNA into the genome of a target cell, mutations may be created in the cargo DNA upon integration into the target cell genome. As shown in FIG. 6, cargo DNA delivered into cells by a lentivirus having a M-RT had a higher nucleic acid substitution rate compared the cargo DNA delivered into cells by a lentivirus having a WT-RT. Sequence data did not show a change in error bias between the delivered cargo DNA. These data showed that use of M-RT does not affect bias but does increase the mutation rate overall.

Similar experiments were conducted comparing M-RT with D-RT (M230I/Y501W) mutant. Briefly, HCC827 cells (harboring the EGFR^{del746-750} mutation) expressing TET3G were transduced with lentiviral particles generated from pLVX-TRE3G-EGFR^{del746-750}-puro packaged with either M-RT or D-RT at an MOI of ˜0.3. Transduced cells were selected with puromycin, then 2 million cells were seeded in 15-cm dishes and selected with 100 nM Osimertinib or 1 μM Gefitinib in the presence of 1 mg/ml doxycycline. On appearance of resistant colonies staining with crystal violet was performed. Colonies from each plate were quantified and the use of D-RT in the LentiMutate method increased the number of inhibitor resistant colonies (FIG. 5D).

### Example 4

LentiMutate was validated using on a different cancer and drug—chronic myeloid leukemia (CML), which harbors the BCR-ABL1 fusion gene/protein and is sensitive to imatinib Mutations in the kinase domain of BCR-ABL1 can promote resistance to imatinib. For this example, the K562 CML cell line was used as it harbored the BCR-ABL1 fusion gene/protein and was highly sensitive to BCR-ABL1 inhibitors. The LentiMutate method as described in the examples above was performed using BCR-ABL1 (p210) and imatinib.

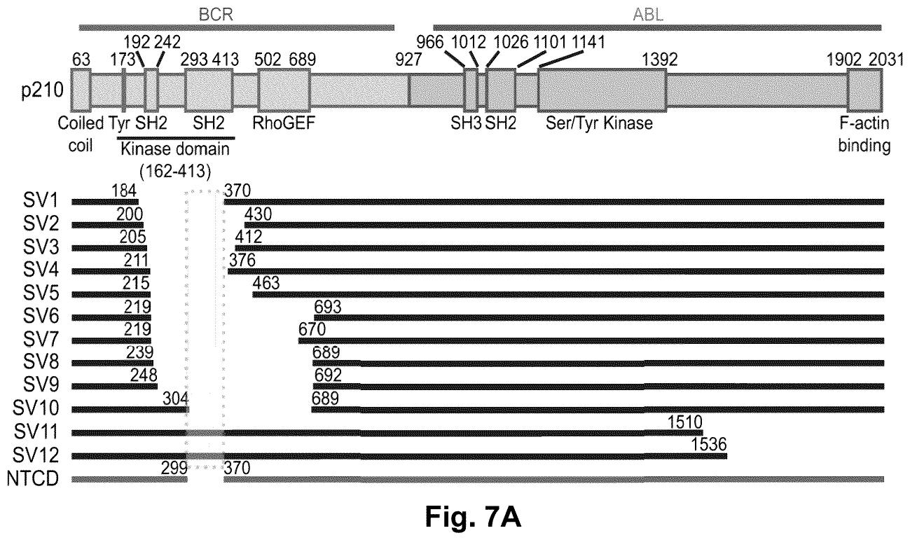

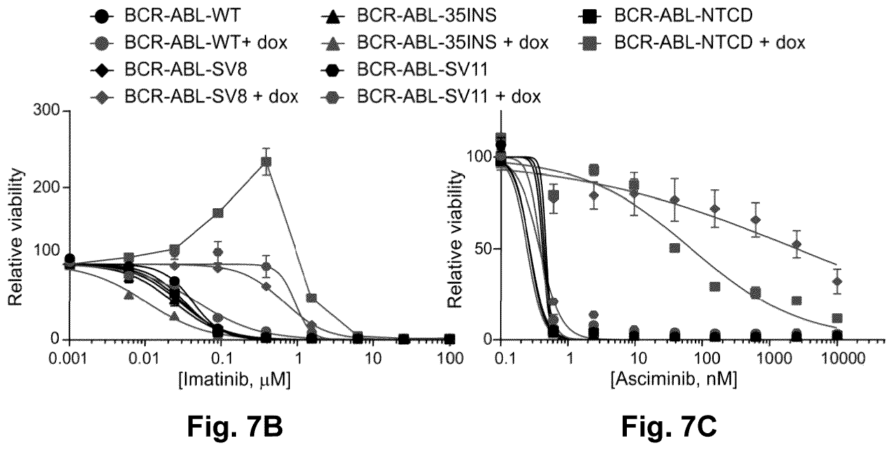

Imatinib resistant cells readily emerged and harbored some of the previously identified imatinib-resistance mutations, such as M4721, E494K, and E255K. However, also identified were numerous deletions (structural variants, or SVs) in BCR-ABL1 outside the ABL1 kinase domain that were strongly enriched and dominated the population of these imatinib resistant cells (FIG. 7A).

LentiMutate may create large and small indels because the HIV-1 RT is known to switch templates and have low processivity. In comparison to other techniques used to identify resistance-conferring mutations, the ability of LentiMutate to create indels and recover them efficiently using deep sequencing was a unique advantage. Three of these deletion mutations directly identified by LentiMutate, as well as a 216-nucleotide region in BCR shared by multiple deletion mutations, referred to as the N-Terminus Consensus Deletion (NTCD), were tested for drug resistance in K562 cells. As a comparison, also tested was the 35INS variant, a BCR-ABL variant detected in patient samples that truncates BCR-ABL at the end of the kinase domain, producing a kinase dead protein, due to the insertion and usage of a 35 bp sequence in an intron that acts as a novel splicing site. This deletion mutant was originally believed to confer resistance to imatinib but subsequently shown not to promote resistance. All the deletions identified by LentiMutate, as well as the NTCD, but not 35INS, conferred resistance to imatinib, an ATP-site directed ABL1 inhibitor (FIG. 7B). By contrast, while deletions in BCR (−SV8 and −NTCD) conferred resistance to the allosteric ABL1 inhibitor asciminib, deletions in ABL1 (−SV11) did not (FIG. 7C).

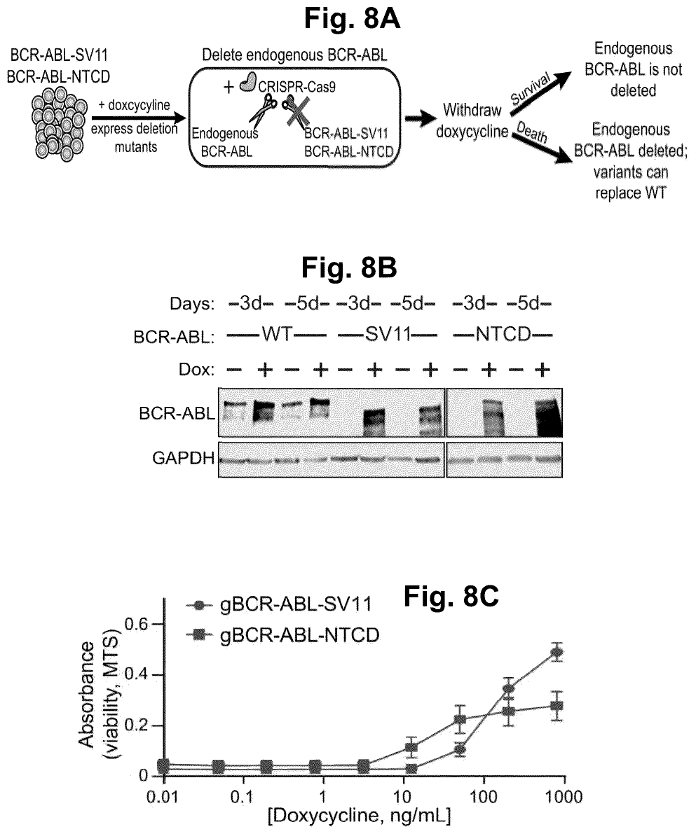

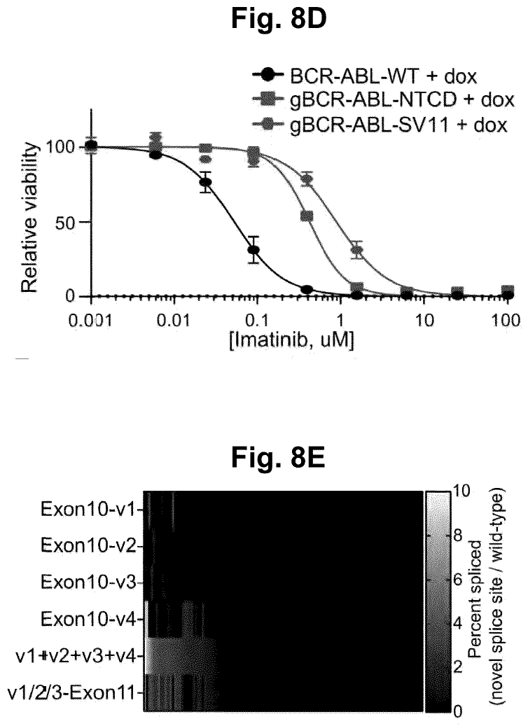

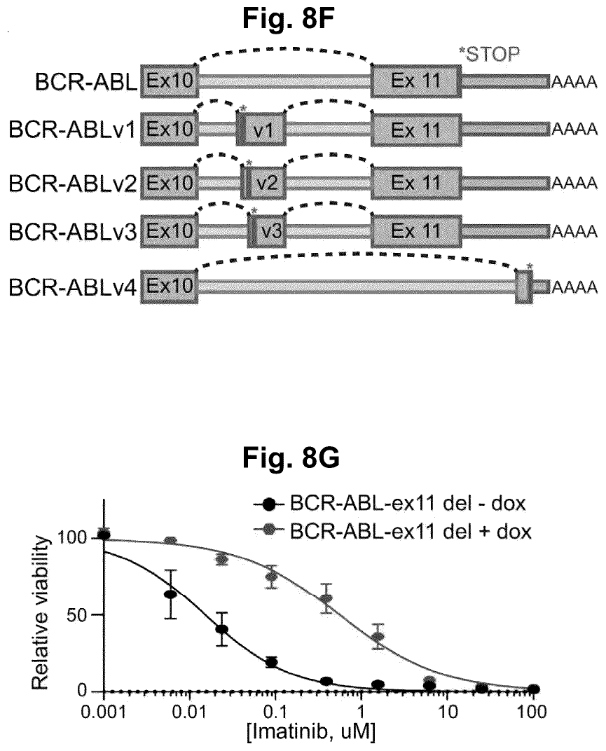

BCR-ABL1 is known to oligomerize when signaling. To rule out artifacts that might occur because the deletion mutants were expressed in the presence of WT BCR-ABL1, endogenous BCR-ABL1 was eliminated so that only the BCR-ABL1 deletion mutants were expressed. To do this, the endogenous BCR-ABL1 was deleted while expressing the exogenous, doxycycline-inducible BCR-ABL1 deletion mutants (FIG. 8A). Single cell clones were identified from the population that lacked WT BCR-ABL1 and loss expression of exogenous BCR-ABL1 (FIG. 8B) and viability (FIG. 8C) after withdrawal of doxycycline. Additionally, these cells are resistant to imatinib (FIG. 8D), providing formal proof that these deletion mutants alone can promote resistance to imatinib.

Numerous studies have examined CML patient samples for mutations in BCR-ABL1 that may confer resistance to BCR-ABL1 inhibitors; however, these studies tend to sequence only the kinase domain in ABL1. To search for events in CML patients that may produce proteins similar to the BCR-ABL1 deletion mutants identified by LentiMutate, Exome-Seq and RNA-seq data were analyzed from 120 CML patients that were either newly diagnosed or had relapsed on BCR-ABL1 inhibitors. Using the Exome-Seq dataset no mutations were identified that would create deletions similar to those identified by LentiMutate. In contrast, in the RNA-seq dataset, split reads were identified in 27.5% of patients that demonstrated either the inclusion of cryptic exons in the intron between the canonical exons 10 and 11 (v1, v2, v3) or alternative splicing between exons 10 and 11 (v4) in ABL1 (FIG. 8E). All four of these splice variants would be expected to truncate BCR-ABL1 (FIG. 8F) in a manner highly similar to the C-terminal BCR-ABL1 deletions identified by LentiMutate. The expected sequences of novel BCR-ABL splicing isoforms identified in patient samples are provided below where Exon10 is in normal text, Exon11 is italicized, and v1, v2, v3, and v4 are underlined:

“Wild-type BCR ABL”, canonical transcript Exon10-Exon11 (SEQ ID NO: 9):
AAGACGAGGACCTCCAGGAGAGCTGCAGAGCACAGAGACACCACTGACGTGCCTGAGATGCCTCA
CTCCAAGGGCCAGGGAGAGAGCGATCCTCTGGACCATGAGCCTGCCGTGTCTCCATTGCTCCCTC
GAAAAGAGCGAGGTCCCCCGGAGGGCGGCCTGAATGAAGATGAGCGCC
Exon10-Exonv1-Exon11 (SEQ ID NO: 10):
AAGACGAGGACCTCCAGGAGAGCTGCAGAGCACAGAGACACCACTGACGTGCCTGAGATGCCTCA
CTCCAAGGGCCAGGGAGAGAGCGGTATACCCAAGACTGGGTAATTTATAAAGGAAAGAGGTTTCA
CTGACTCACAGTTCCACATGGCTGGGGAGGCCTCACAATCATGGCTGAAGGTGAATGAGGAGCAA
GGTCACATCTTACTTGGCGGCAGGCAAGAGAGCTTGTGCAGGGGAAGTCCGCTTTATAAAACCAT
CAGATCTCGTGAGACTTATTCACTACCACGAGAATGTGGGAGAAACCTCCCCATGATTCATTGAT
CTCCACCTGACCCCACCGTTGACACGTGGGGCTTATTACAATTCAAGATCCTCTGGACCATGAGC
CTGCCGTGTCTCCATTGCTCCCTCGAAAAGAGCGAGGTCCCCCGGAGGGCGGCCTGAATGAAGAT
GAGCGCC
Exon10-Exonv2-Exon11 (SEQ ID NO: 11):
AAGACGAGGACCTCCAGGAGAGCTGCAGAGCACAGAGACACCACTGACGTGCCTGAGATGCCTCA
CTCCAAGGGCCAGGGAGAGAGCGACTGGGTAATTTATAAAGGAAAGAGGTTTCACTGACTCACAG
TTCCACATGGCTGGGGAGGCCTCACAATCATGGCTGAAGGTGAATGAGGAGCAAGGTCACATCTT
ACTTGGCGGCAGGCAAGAGAGCTTGTGCAGGGGAAGTCCGCTTTATAAAACCATCAGATCTCGTG
AGACTTATTCACTACCACGAGAATGTGGGAGAAACCTCCCCATGATTCATTGATCTCCACCTGAC
CCCACCGTTGACACGTGGGGCTTATTACAATTCAAGATCCTCTGGACCATGAGCCTGCCGTGTCT
CCATTGCTCCCTCGAAAAGAGCGAGGTCCCCCGGAGGGCGGCCTGAATGAAGATGAGCGCC
Exon10-Exonv3-Exon11 (SEQ ID NO: 12):
AAGACGAGGACCTCCAGGAGAGCTGCAGAGCACAGAGACACCACTGACGTGCCTGAGATGCCTCA
CTCCAAGGGCCAGGGAGAGAGCGTTCCACATGGCTGGGGAGGCCTCACAATCATGGCTGAAGGTG
AATGAGGAGCAAGGTCACATCTTACTTGGCGGCAGGCAAGAGAGCTTGTGCAGGGGAAGTCCGCT
TTATAAAACCATCAGATCTCGTGAGACTTATTCACTACCACGAGAATGTGGGAGAAACCTCCCCA
TGATTCATTGATCTCCACCTGACCCCACCGTTGACACGTGGGGCTTATTACAATTCAAGATCCTC
TGGACCATGAGCCTGCCGTGTCTCCATTGCTCCCTCGAAAAGAGCGAGGTCCCCCGGAGGGCGGC
CTGAATGAAGATGAGCGCC
Exon10-Exon v4 (part of Exon11) (SEQ ID NO: 13):
AAGACGAGGACCTCCAGGAGAGCTGCAGAGCACAGAGACACCACTGACGTGCCTGAGATGCCTCA
CTCCAAGGGCCAGGGAGAGAGCGAAATGGTTTCCTCTGGATCGTTTTATGCGGTTCTTACAGCAC
ATCACCTCTTTGCCCCCGACGGCTGTGACGCAGCCGGAGGGAGGCACTAGTCACCGACAGCGGCC
TTGAAGACAGAGCAAAGCGCCCACCCAGGTCCCCCG

To determine if these splice variants of BCR-ABL1 could confer resistance to imatinib, a BCR-ABL1 construct (BCR-ABL1-ex11del) was generated that lacks exon 11 to mimic these novel BCR-ABL1 isoforms. Expression of BCR-ABL1-ex11del was able to confer resistance to imatinib (FIG. 8G). Of note, alternative splicing has been documented to generate a truncated androgen receptor (AR) isoform, known as AR-V7, which was believed to promote clinical resistance to enzalutamide and abiraterone, analogous to the BCR-ABL1 isoforms identified in this study.

### Example 5

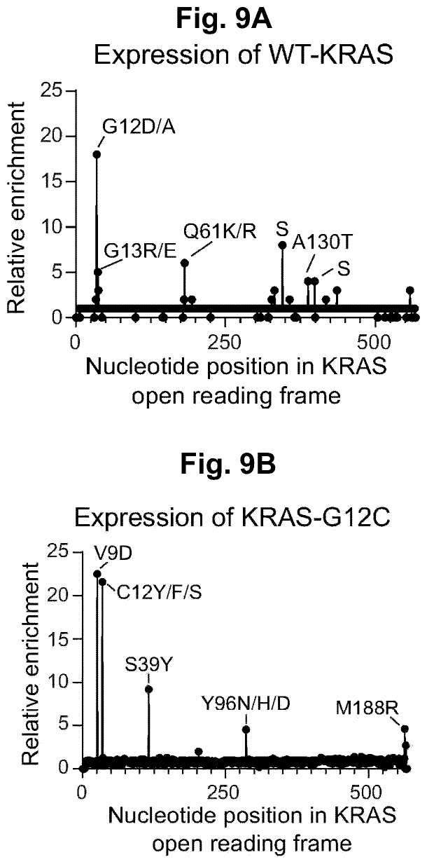

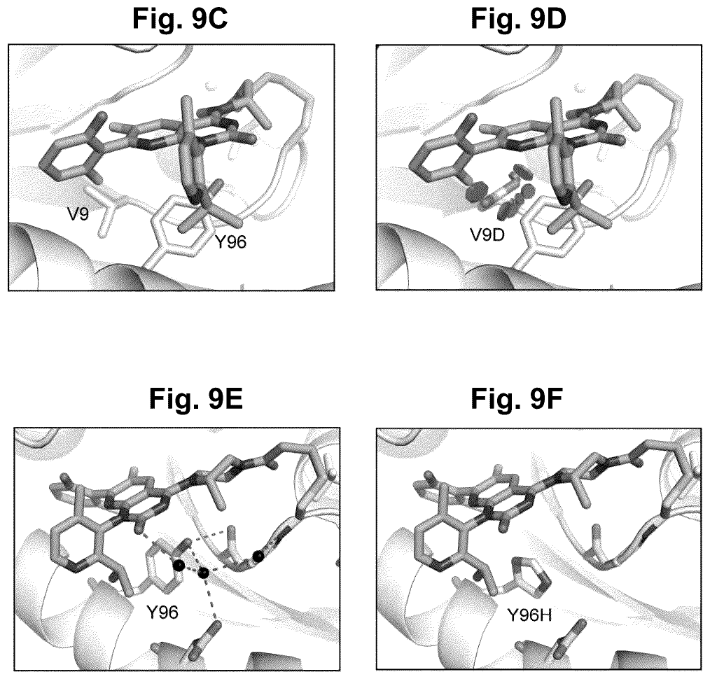

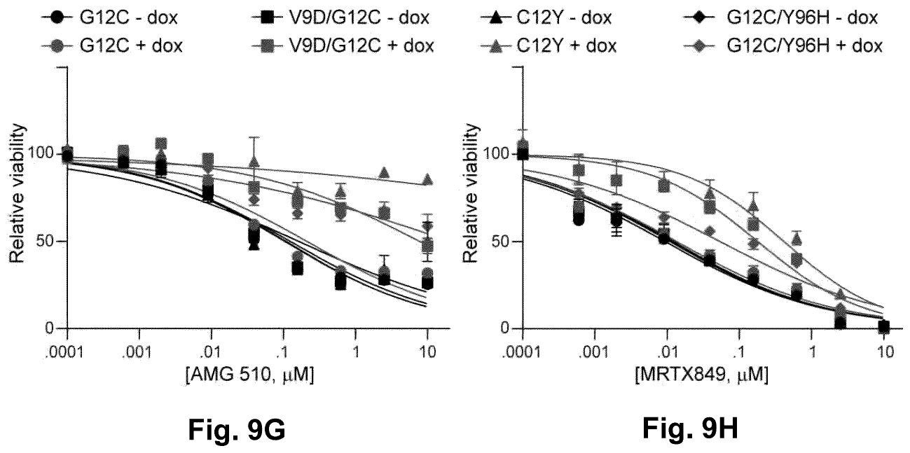

To demonstrate the potential ability of LentiMutate to prospectively identify resistance mutations before they occur in patient's tumors, the KRAS-G12C inhibitor AMG 510 was studied in this example. KRAS mutations in NSCLC patients can be heterozygous; therefore, LentiMutate was performed according to the methods described in the examples above using either WT KRAS or KRAS-G12C and AMG 510 in the KRAS-G12C mutant and addicted NSCLC cell line H358. LentiMutate using WT KRAS identified numerous KRAS mutations that have been shown to be oncogenic, such as KRAS-G12D and KRAS-Q61R (FIG. 9A). By contrast, LentiMutate using KRAS-G12C primarily identified point mutations that are predicted to alter drug binding, such as various mutations in cysteine 12, as well as mutations in the AMG 510 binding pocket, such as V9D and Y96N/H/D (FIG. 9B).

Codon 9 was structurally conserved as a hydrophobic residue within the RAS superfamily and the V9D mutation altered the electrostatic environment, resulting in clashes between the aspartate, Y96 and AMG 510 (FIGS. 9C and 9D). Y96 participated in hydrophobic interactions and a network of polar contacts that supported ligand binding, including interactions between the hydroxyl and backbone of G10 and interactions with a network of waters. The Y96H mutation removed these polar contacts and electron density and was predicted to impair ligand binding (FIGS. 9E and 9F). Expression of these mutants conferred resistance to AMG 510 (FIG. 9G), as well as a different KRAS-G12C inhibitor, MRTX849 (FIG. 9H).

What is claimed is:

1. A method of screening for one or more mutations in a nucleic acid sequence encoding a protein in a target cell; wherein the one or more mutations conveys a phenotypic property to the protein, comprising:
(a) transducing the target cell with at least one lentiviral particle comprising a pol gene nucleic acid sequence encoding a reverse transcriptase sequence comprising a M230I and a Y501W mutation with reference to SEQ ID NO: 1;
(b) contacting the target cell with a biomolecule after step (a), to select for the target cell with the phenotypic property in a target cell population;
(c) obtaining the nucleic acid sequence from the target cell with the phenotypic property; and
(d) screening for one or more mutations that convey the phenotypic property, wherein screening comprises comparing the nucleic acid sequence encoding the protein from the target cell with the phenotypic property, to a control nucleic acid sequence, wherein the control nucleic acid sequence encodes the protein without the one or more mutations.

2. The method of claim 1, wherein the reverse transcriptase sequence comprises an amino acid sequence as set forth in SEQ ID NO: 14, or a sequence at least about 80% identical thereto.

3. The method of claim 1, wherein the nucleic acid sequence encoding the protein is a genomic nucleic acid sequence.

4. The method of claim 1, wherein the nucleic acid sequence encoding the protein is a heterologous nucleic acid sequence.

5. The method of claim 4, wherein the at least one lentiviral particle further comprises the heterologous nucleic acid sequence.

6. The method of claim 5, wherein the lentiviral particle further comprises an inducible promoter operatively linked to the heterologous nucleic acid sequence.

7. The method of claim 6, wherein the inducible promoter is a tetracycline-regulated promoter.

8. The method of claim 1, wherein the nucleic acid sequence encoding for the protein encodes for 2B4, 4-1 BB, 4-1BBL, A33, adenosine A2a receptor, Akt, ALK, Androgen receptor, Ang-1, Ang-2, Annexin A3, Aurora A, Aurora B, B7-H3, B7-H4, Bcl-2, Bcr-Abl, BRAF, BTK, BTLA, BTN2A1, CA-125, CAIX, CCR4, CD105/endoglin, CD109, CD123, CD155, CD16, CD160, CD19, CD20, CD200, CD200R, CD22, CD24, CD25, CD27, CD28, CD30, CD33, CD36, CD37, CD38, CD40, CD40L, CD47, CD48, CD52, CD70, CD79b, CD80, CD86, CD96, CDK4, CDK6, CDK9, CEA, CEACAM1, ChK1, ChK2, c-KIT, c-Met/HGFR, COX2, CSF-1R, CSF2, CTLA-4, CXCR2, CXCR4, DDR2, DLL3, DLL4, DNAM-1, DR5, EGFR, EpCAM, EPHA3, EphB4, ERK1, ERK2/p38 MAPK, FAK, FAP, FGF-2, FGFR1, FGFR2, FGFR3, FGFR4, Flt-3, Gal-9, GITR, GITRL, Glypican-3, HDAC1, HDAC2, HDAC3, HDAC4, HDAC5, HDAC7, HDAC8, HDAC9, HER2, HER3, HER4/ERBB4, HGF, HHLA2, HIF-1α, HSP27, HSP90, HVEM, ICOS, ICOS Ligand, IDO, IGF1R, IL-13, IL-6, JAK1, JAK2, JAK3, KRAS, LAG-3, LIGHT, MDM2, MEK1, MEK2, MMP-1, MMP-10, MMP-11, MMP-13, MMP-2, MMP-7, MMP-9, mTOR, Mucin 1, Myc, NF-κB, NKG2A, NRAS, NTRK1, NTRK2, NTRK3, OX40, OX40L, p53, PAF, PARP1, PARP2, PD1, PDGFR-α, PDGFR-β, PD-L1, PD-L2, PI3Kα, PI3Kβ, PI3Kγ, PI3Kδ, PIM1, PIM3, PSMA, PTEN, RAF-1, RANKL, RET, S100A4, SIRPα, SLAMF7, SMO, Src, STAT3, STEAP-1, Syk, TDO, TGFβ, Tie-2, TIGIT, TIM-3, TLR8, TMIGD2, TNF-α, Toll-like receptor 3, TRAIL, TRAILR1, TROP-2, VEGF, VEGF-C, VEGFR-1, VEGFR-2, VEGFR-3, VISTA, γ-secretase, or any combination thereof.

9. The method of claim 1, wherein the mutation comprises a substitution of a base, an insertion of a base, an insertion of one or more bases, a deletion of one or more bases, or any combination thereof.

10. The method of claim 9, wherein the substitution of a base comprises substituting a C for a G, an A for a C, a T for a G, a G for a C, an A for a T, a T for an A, a G for a T, a T for a C, an A for a G, a C for an A, a C for a T, a G for an A, or any combination thereof.

11. The method of claim 9, wherein the mutation comprises an indel.

12. The method of claim 1, wherein the phenotypic property is target cell viability in presence of the biomolecule.

13. The method of claim 12, wherein the biomolecule comprises a peptide, a protein, an enzyme, an antibody, an aptamer, DNA, RNA, siRNA, an oligonucleotide, a small molecule, or any combination thereof.

14. The method of claim 1, wherein the sequence of the nucleic acid is obtained by Sanger sequencing, pyrosequencing, reversible terminator chemistry, sequencing by ligation, H+ Ion sensitive transistor, nanopore sequencing, next generation sequencing, or any combination thereof.

15. The method of claim 1, wherein the biomolecule comprises a peptide, a protein, an enzyme, an antibody, an aptamer, DNA, RNA, siRNA, an oligonucleotide, a small molecule, a drug, or any combination thereof.

16. A composition for screening for one or more mutations conveying a phenotypic property in a protein comprising, at least a lentiviral particle comprising:
(a) a pol gene nucleic acid sequence encoding a reverse transcriptase sequence comprising a M230I and a Y501W mutation with reference to SEQ ID NO: 1; and
(b) a heterologous nucleic acid sequence encoding the protein.

17. The composition of claim 16, wherein the reverse transcriptase comprises an amino acid sequence as set forth in SEQ ID NO: 14, or a sequence at least about 80% identical thereto.

18. A method of screening for one or more genomic mutations in a target cell, wherein the one or more genomic mutations confers drug-resistance to a drug, to the target cell, the method comprising:
(a) transducing the target cell with at least one lentiviral particle comprising a pol gene nucleic acid sequence encoding a reverse transcriptase sequence comprising a M230I and a Y501W mutation with reference to SEQ ID NO: 1;
(b) contacting the target cell with the drug after step (a), to select for the target cell with the drug resistance, in a target cell population;
(c) obtaining the genomic sequence from the target cell with drug resistance;
(d) screening for one or more mutations that confer the drug resistance, wherein screening comprises comparing the genomic sequence of the target cell with the drug resistance, to a control genomic sequence obtained from a cell without drug resistance.

19. The method of claim 18, wherein the reverse transcriptase comprises an amino acid sequence as set forth in SEQ ID NO: 14, or a sequence at least about 80% identical thereto.

20. The method of claim 19, wherein the drug comprises a drug for treatment of a cancer.

21. The method of claim 20, wherein the cancer comprises breast cancer, prostate cancer, lymphoma, skin cancer, pancreatic cancer, colon cancer, melanoma, malignant melanoma, ovarian cancer, brain cancer, primary brain carcinoma, head-neck cancer, glioma, glioblastoma, liver cancer, bladder cancer, non-small cell lung cancer, head or neck carcinoma, breast carcinoma, ovarian carcinoma, lung carcinoma, small-cell lung carcinoma, Wilms' tumor, cervical carcinoma, testicular carcinoma, bladder carcinoma, pancreatic carcinoma, stomach carcinoma, colon carcinoma, prostatic carcinoma, genitourinary carcinoma, thyroid carcinoma, esophageal carcinoma, myeloma, multiple myeloma, adrenal carcinoma, renal cell carcinoma, endometrial carcinoma, adrenal cortex carcinoma, malignant pancreatic insulinoma, malignant carcinoid carcinoma, choriocarcinoma, mycosis fungoides, malignant hypercalcemia, cervical hyperplasia, leukemia, acute lymphocytic leukemia, chronic lymphocytic leukemia, acute myelogenous leukemia, chronic myelogenous leukemia, chronic granulocytic leukemia, acute granulocytic leukemia, hairy cell leukemia, neuroblastoma, rhabdomyosarcoma, Kaposi's sarcoma, polycythemia vera, essential thrombocytosis, Hodgkin's disease, non-Hodgkin's lymphoma, soft-tissue sarcoma, mesothelioma, osteogenic sarcoma, primary macroglobulinemia, retinoblastoma, or any combination thereof.

22. The method of claim 18, wherein the one or more mutation is in a drug target protein comprising 2B4, 4-1 BB, 4-1 BBL, A33, adenosine A2a receptor, Akt, ALK, Androgen receptor, Ang-1, Ang-2, Annexin A3, Aurora A, Aurora B, E7-H3, E7-H4, Bcl-2, Bcr-Abl, BRAF, BTK, BTLA, BTN2A1, CA-125, CAIX, CCR4, CD105/endoglin, CD109, CD123, CD155, CD16, CD160, CD19, CD20, CD200, CD200R, CD22, CD24, CD25, CD27, CD28, CD30, CD33, CD36, CD37, CD38, CD40, CD40L, CD47, CD48, CD52, CD70, CD79b, CD80, CD86, CD96, CDK4, CDK6, CDK9, CEA, CEACAM1, ChK1, ChK2, c-KIT, c-Met/HGFR, COX2, CSF-1R, CSF2, CTLA-4, CXCR2, CXCR4, DDR2, DLL3, DLL4, DNAM-1, DR5, EGFR, EpCAM, EPHA3, EphB4, ERK1, ERK2/p38 MAPK, FAK, FAP, FGF-2, FGFR1, FGFR2, FGFR3, FGFR4, Flt-3, Gal-9, GITR, GITRL, Glypican-3, HDAC1, HDAC2, HDAC3, HDAC4, HDAC5, HDAC7, HDAC8, HDAC9, HER2, HER3, HER4/ERBB4, HGF, HHLA2, HIF-1α, HSP27, HSP90, HVEM, ICOS, ICOS Ligand, IDO, IGF1R, IL-13, IL-6, JAK1, JAK2, JAK3, KRAS, LAG-3, LIGHT, MDM2, MEK1, MEK2, MMP-1, MMP-10, MMP-11, MMP-13, MMP-2, MMP-7, MMP-9, mTOR, Mucin 1, Myc, NF-κB, NKG2A, NRAS, NTRK1, NTRK2, NTRK3, OX40, OX40L, p53, PAF, PARP1, PARP2, PD1, PDGFR-α, PDGFR-β, PD-L1, PD-L2, PI3Kα, PI3Kβ, PI3Kγ, PI3Kδ, PIM1, PIM3, PSMA, PTEN, RAF-1, RANKL, RET, S100A4, SIRPα, SLAMF7, SMO, Src, STAT3, STEAP-1, Syk, TDO, TGFβ, Tie-2, TIGIT, TIM-3, TLR8, TMIGD2, TNF-α, Toll-like receptor 3, TRAIL, TRAILR1, TROP-2, VEGF, VEGF-C, VEGFR-1, VEGFR-2, VEGFR-3, VISTA, γ-secretase, or any combination thereof.


SEQUENCE LISTING (14 sequences)

SEQ ID NO: 1 - protein, 560 aa, synthetic construct
  REGION 1..560: note = Synthetic Construct; WT-RT
PISPIETVPVKLKPGMDGPKVKQWPLTEEKIKALVEICTEMEKEGKISKIGPENPYNTPVFAIKKKDSTKWRKLVDFRELNKRTQDFWEVQLGIPHPAGLKKKKSVTVLDVGDAYFSVPLDEDFRKYTAFTIPSINNETPGIRYQYNVLPQGWKGSPAIFQSSMTKILEPFRKQNPDIVIYQYMDDLYVGSDLEIGQHRTKIEELRQHLLRWGLTTPDKKHQKEPPFLWMGYELHPDKWTVQPIVLPEKDSWTVNDIQKLVGKLNWASQIYPGIKVRQLCKLLRGTKALTEVIPLTEEAELELAENREILKEPVHGVYYDPSKDLIAEIQKQGQGQWTYQIYQEPFKNLKTGKYARMRGAHTNDVKQLTEAVQKITTESIVIWGKTPKFKLPIQKETWETWWTEYWQATWIPEWEFVNTPPLVKLWYQLEKEPIVGAETFYVDGAANRETKLGKAGYVTNRGRQKVVTLTDTTNQKTELQAIYLALQDSGLEVNIVTDSQYALGIIQAQPDQSESELVNQIIEQLIKKEKVYLAWVPAHKGIGGNEQVDKLVSAGIRKVL

SEQ ID NO: 2 - protein, 560 aa, synthetic construct
  REGION 1..560: note = Synthetic Construct; M230I
PISPIETVPVKLKPGMDGPKVKQWPLTEEKIKALVEICTEMEKEGKISKIGPENPYNTPVFAIKKKDSTKWRKLVDFRELNKRTQDFWEVQLGIPHPAGLKKKKSVTVLDVGDAYFSVPLDEDFRKYTAFTIPSINNETPGIRYQYNVLPQGWKGSPAIFQSSMTKILEPFRKQNPDIVIYQYMDDLYVGSDLEIGQHRTKIEELRQHLLRWGLTTPDKKHQKEPPFLWIGYELHPDKWTVQPIVLPEKDSWTVNDIQKLVGKLNWASQIYPGIKVRQLCKLLRGTKALTEVIPLTEEAELELAENREILKEPVHGVYYDPSKDLIAEIQKQGQGQWTYQIYQEPFKNLKTGKYARMRGAHTNDVKQLTEAVQKITTESIVIWGKTPKFKLPIQKETWETWWTEYWQATWIPEWEFVNTPPLVKLWYQLEKEPIVGAETFYVDGAANRETKLGKAGYVTNRGRQKVVTLTDTTNQKTELQAIYLALQDSGLEVNIVTDSQYALGIIQAQPDQSESELVNQIIEQLIKKEKVYLAWVPAHKGIGGNEQVDKLVSAGIRKVL

SEQ ID NO: 3 - DNA, 25 bp, synthetic construct
  misc_feature 1..25: note = Synthetic Construct; gBCR-ABL-NTCD-1
  source 1..25: mol_type = other DNA
caccgctactccccccggagttttg

SEQ ID NO: 4 - DNA, 25 bp, synthetic construct
  misc_feature 1..25: note = Synthetic Construct; gBCR-ABL-NTCD-2
  source 1..25: mol_type = other DNA
caccgagggagagcggcttttgtcc

SEQ ID NO: 5 - DNA, 25 bp, synthetic construct
  misc_feature 1..25: note = Synthetic Construct; gBCR-ABL-NTCD-3
  source 1..25: mol_type = other DNA
caccgagaggagaagtcctcctcgc

SEQ ID NO: 6 - DNA, 25 bp, synthetic construct
  misc_feature 1..25: note = Synthetic Construct; gBCR-ABL-sv11
  source 1..25: mol_type = other DNA
caccgaaggagctgctgcgtttggg

SEQ ID NO: 7 - DNA, 567 bp, synthetic construct
  misc_feature 1..567: note = Synthetic Construct; KRAS-WT
  source 1..567: mol_type = other DNA
atgacagaatataaactggtagtagtaggtgcaggtggagttggaaaatccgctcttacaattcaactcatccaaaaccacttcgtcgatgagtacgaccccactatcgaagacagttatagaaaacaggtcgttatcgacggcgagacgtgccttctcgacatcctggataccgccggccaggaagaatatagcgccatgcgggatcaatatatgcgtacaggcgaagggttcctgtgcgtgttcgcaattaacaacacgaagtccttcgaggacatccatcactaccgggagcagatcaagcgtgtgaaagatagcgaggacgtcccgatggtgcttgtgggcaacaagtgcgatctcccaagccgaactgtggataccaagcaagcacaagatctggctcggtcctacggcatccccttcatcgagacgtctgccaaaacgaggcaaggcgtagacgacgcattttacacgctggtgagagagatccgcaagcacaaggagaaaatgtctaaggacggaaagaagaagaagaagaagagcaaaacgaaatgcgtgatcatgtag

SEQ ID NO: 8 - DNA, 567 bp, synthetic construct
  misc_feature 1..567: note = Synthetic Construct; KRAS-G12C
  source 1..567: mol_type = other DNA
atgacagaatataaattggtcgttgttggagcttgtggtgtcgggaaatctgcacttaccattcaattgatacaaaaccacttcgtagatgagtacgaccctaccattgaagactcttatagaaaacaggttgtgatagacggggagacatgcctgcttgacatcctggatactgcgggacaggaagaatattcagccatgcgcgatcaatatatgcggacaggcgaagggttcctgtgcgtgttcgctattaacaacacaaagagcttcgaggacatacatcactaccgtgagcagatcaagcgggtgaaagattcagaggacgtgcccatggttctcgtgggcaacaagtgcgaccttcccagtaggactgtcgatacgaagcaagcccaagatctggcccggagctacggtatcccattcatcgagactagcgctaaaactcggcaaggcgtggacgacgcattttacactctcgtccgcgagatcagaaagcacaaggagaaaatgtctaaggacggcaagaagaagaagaagaaatccaaaaccaaatgcgtgatcatgtga

SEQ ID NO: 9 - DNA, 178 bp, synthetic construct
  misc_feature 1..178: note = Synthetic Construct; Wild-type BCR ABL, canonical transcript Exon10-Exon11
  source 1..178: mol_type = other DNA
aagacgaggacctccaggagagctgcagagcacagagacaccactgacgtgcctgagatgcctcactccaagggccagggagagagcgatcctctggaccatgagcctgccgtgtctccattgctccctcgaaaagagcgaggtcccccggagggcggcctgaatgaagatgagcgcc

SEQ ID NO: 10 - DNA, 462 bp, synthetic construct
  misc_feature 1..462: note = Synthetic Construct; Exon10-Exonv1-Exon11
  source 1..462: mol_type = other DNA
aagacgaggacctccaggagagctgcagagcacagagacaccactgacgtgcctgagatgcctcactccaagggccagggagagagcggtatacccaagactgggtaatttataaaggaaagaggtttcactgactcacagttccacatggctggggaggcctcacaatcatggctgaaggtgaatgaggagcaaggtcacatcttacttggcggcaggcaagagagcttgtgcaggggaagtccgctttataaaaccatcagatctcgtgagacttattcactaccacgagaatgtgggagaaacctccccatgattcattgatctccacctgaccccaccgttgacacgtggggcttattacaattcaagatcctctggaccatgagcctgccgtgtctccattgctccctcgaaaagagcgaggtcccccggagggcggcctgaatgaagatgagcgcc

SEQ ID NO: 11 - DNA, 451 bp, synthetic construct
  misc_feature 1..451: note = Synthetic Construct; Exon10-Exonv2-Exon11
  source 1..451: mol_type = other DNA
aagacgaggacctccaggagagctgcagagcacagagacaccactgacgtgcctgagatgcctcactccaagggccagggagagagcgactgggtaatttataaaggaaagaggtttcactgactcacagttccacatggctggggaggcctcacaatcatggctgaaggtgaatgaggagcaaggtcacatcttacttggcggcaggcaagagagcttgtgcaggggaagtccgctttataaaaccatcagatctcgtgagacttattcactaccacgagaatgtgggagaaacctccccatgattcattgatctccacctgaccccaccgttgacacgtggggcttattacaattcaagatcctctggaccatgagcctgccgtgtctccattgctccctcgaaaagagcgaggtcccccggagggcggcctgaatgaagatgagcgcc

SEQ ID NO: 12 - DNA, 409 bp, synthetic construct
  misc_feature 1..409: note = Synthetic Construct; Exon10-Exonv3-Exon11
  source 1..409: mol_type = other DNA
aagacgaggacctccaggagagctgcagagcacagagacaccactgacgtgcctgagatgcctcactccaagggccagggagagagcgttccacatggctggggaggcctcacaatcatggctgaaggtgaatgaggagcaaggtcacatcttacttggcggcaggcaagagagcttgtgcaggggaagtccgctttataaaaccatcagatctcgtgagacttattcactaccacgagaatgtgggagaaacctccccatgattcattgatctccacctgaccccaccgttgacacgtggggcttattacaattcaagatcctctggaccatgagcctgccgtgtctccattgctccctcgaaaagagcgaggtcccccggagggcggcctgaatgaagatgagcgcc

SEQ ID NO: 13 - DNA, 231 bp, synthetic construct
  misc_feature 1..231: note = Synthetic Construct; Exon10-Exon v4 (part of Exon11)
  source 1..231: mol_type = other DNA
aagacgaggacctccaggagagctgcagagcacagagacaccactgacgtgcctgagatgcctcactccaagggccagggagagagcgaaatggtttcctctggatcgttttatgcggttcttacagcacatcacctctttgcccccgacggctgtgacgcagccggagggaggcactagtcaccgacagcggccttgaagacagagcaaagcgcccacccaggtcccccg

SEQ ID NO: 14 - protein, 560 aa, synthetic construct
PISPIETVPVKLKPGMDGPKVKQWPLTEEKIKALVEICTEMEKEGKISKIGPENPYNTPVFAIKKKDSTKWRKLVDFRELNKRTQDFWEVQLGIPHPAGLKKKKSVTVLDVGDAYFSVPLDEDFRKYTAFTIPSINNETPGIRYQYNVLPQGWKGSPAIFQSSMTKILEPFRKQNPDIVIYQYMDDLYVGSDLEIGQHRTKIEELRQHLLRWGLTTPDKKHQKEPPFLWIGYELHPDKWTVQPIVLPEKDSWTVNDIQKLVGKLNWASQIYPGIKVRQLCKLLRGTKALTEVIPLTEEAELELAENREILKEPVHGVYYDPSKDLIAEIQKQGQGQWTYQIYQEPFKNLKTGKYARMRGAHTNDVKQLTEAVQKITTESIVIWGKTPKFKLPIQKETWETWWTEYWQATWIPEWEFVNTPPLVKLWYQLEKEPIVGAETFYVDGAANRETKLGKAGYVTNRGRQKVVTLTDTTNQKTELQAIYLALQDSGLEVNIVTDSQWALGIIQAQPDQSESELVNQIIEQLIKKEKVYLAWVPAHKGIGGNEQVDKLVSAGIRKVL